SEQUENCE LISTING (46 sequences)

SEQ ID NO: 1 - DNA, 268 bp, Homo sapiens
  source 1..268: mol_type = genomic DNA
cacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctgga

SEQ ID NO: 2 - DNA, 53 bp, synthetic construct
  misc_feature 1..53: note = PCR primer
  source 1..53: mol_type = other DNA
ctcggcattcctgctgaaccgctcttccgatctacaggaggtgggggttagac

SEQ ID NO: 3 - DNA, 59 bp, synthetic construct
  misc_feature 1..59: note = PCR primer
  source 1..59: mol_type = other DNA
acactctttccctacacgacgctcttccgatctcgtgattatattcccagggccggtta

SEQ ID NO: 4 - DNA, 59 bp, synthetic construct
  misc_feature 1..59: note = PCR primer
  source 1..59: mol_type = other DNA
acactctttccctacacgacgctcttccgatctacatcgtatattcccagggccggtta

SEQ ID NO: 5 - DNA, 59 bp, synthetic construct
  misc_feature 1..59: note = PCR primer
  source 1..59: mol_type = other DNA
acactctttccctacacgacgctcttccgatctgcctaatatattcccagggccggtta

SEQ ID NO: 6 - DNA, 59 bp, synthetic construct
  misc_feature 1..59: note = PCR primer
  source 1..59: mol_type = other DNA
acactctttccctacacgacgctcttccgatcttggtcatatattcccagggccggtta

SEQ ID NO: 7 - DNA, 59 bp, synthetic construct
  misc_feature 1..59: note = PCR primer
  source 1..59: mol_type = other DNA
acactctttccctacacgacgctcttccgatctcactgttatattcccagggccggtta

SEQ ID NO: 8 - DNA, 59 bp, synthetic construct
  misc_feature 1..59: note = PCR primer
  source 1..59: mol_type = other DNA
acactctttccctacacgacgctcttccgatctattggctatattcccagggccggtta

SEQ ID NO: 9 - DNA, 59 bp, synthetic construct
  misc_feature 1..59: note = PCR primer
  source 1..59: mol_type = other DNA
acactctttccctacacgacgctcttccgatctgatctgtatattcccagggccggtta

SEQ ID NO: 10 - DNA, 59 bp, synthetic construct
  misc_feature 1..59: note = PCR primer
  source 1..59: mol_type = other DNA
acactctttccctacacgacgctcttccgatcttcaagttatattcccagggccggtta

SEQ ID NO: 11 - DNA, 59 bp, synthetic construct
  misc_feature 1..59: note = PCR primer
  source 1..59: mol_type = other DNA
acactctttccctacacgacgctcttccgatctctgatctatattcccagggccggtta

SEQ ID NO: 12 - DNA, 59 bp, synthetic construct
  misc_feature 1..59: note = PCR primer
  source 1..59: mol_type = other DNA
acactctttccctacacgacgctcttccgatctaagctatatattcccagggccggtta

SEQ ID NO: 13 - DNA, 59 bp, synthetic construct
  misc_feature 1..59: note = PCR primer
  source 1..59: mol_type = other DNA
acactctttccctacacgacgctcttccgatctgtagcctatattcccagggccggtta

SEQ ID NO: 14 - DNA, 59 bp, synthetic construct
  misc_feature 1..59: note = PCR primer
  source 1..59: mol_type = other DNA
acactctttccctacacgacgctcttccgatcttacaagtatattcccagggccggtta

SEQ ID NO: 15 - DNA, 20 bp, synthetic construct
  misc_feature 1..20: note = PCR primer
  source 1..20: mol_type = other DNA
ctgccgtctctctcctgagt

SEQ ID NO: 16 - DNA, 20 bp, synthetic construct
  misc_feature 1..20: note = PCR primer
  source 1..20: mol_type = other DNA
gtgggcttgtactcggtcat

SEQ ID NO: 17 - RNA, 80 nt, synthetic construct
  misc_feature 1..80: note = guide RNA
  source 1..80: mol_type = other RNA
gttttagagctagaaatagcaagttaaaataaggctagtccgttatcaacttgaaaaagtggcaccgagtcggtgctttt

SEQ ID NO: 18 - DNA, 71 bp, Homo sapiens
  source 1..71: mol_type = genomic DNA
taatacttttatcttgtcccctccaccccacagtggggccactagggacaggattggtgacagaaagcccc

SEQ ID NO: 19 - DNA, 52 bp, Homo sapiens
  source 1..52: mol_type = genomic DNA
ttatctgtcccctccaccccacagtggggccactagggaacaggattggtga

SEQ ID NO: 20 - DNA, 22 bp, Homo sapiens
  source 1..22: mol_type = genomic DNA
caggcaggtcctgctttctctg

SEQ ID NO: 21 - DNA, 22 bp, Homo sapiens
  source 1..22: mol_type = genomic DNA
cacagtggggcaagcttctgac

SEQ ID NO: 22 - DNA, 4146 bp, Homo sapiens
  source 1..4146: mol_type = genomic DNA
gccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 23 - DNA, 455 bp, Homo sapiens
  variation 320..338: note = n is a, c, g, or t
  source 1..455: mol_type = genomic DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnngttttagagctagaaatagcaagttaaaataaggctagtccgttatcaacttgaaaaagtggcaccgagtcggtgctttttttctagacccagctttcttgtacaaagttggcatta

SEQ ID NO: 24 - RNA, 99 nt, synthetic construct
  misc_feature 1..99: note = guide RNA
  variation 2..20: note = n is a, c, g, or t
  source 1..99: mol_type = other RNA
gnnnnnnnnnnnnnnnnnnngttttagagctagaaatagcaagttaaaagaagggtagtgggttatcaacttgaaaaagcggcaccgagtcaatacttt

SEQ ID NO: 25 - DNA, 23 bp, Homo sapiens
  source 1..23: mol_type = genomic DNA
gtgaaccgcatcgagctgaaggg

SEQ ID NO: 26 - DNA, 23 bp, Homo sapiens
  source 1..23: mol_type = genomic DNA
ggagcgcaccatcttcttcaagg

SEQ ID NO: 27 - DNA, 23 bp, Homo sapiens
  source 1..23: mol_type = genomic DNA
gtcccctccaccccacagtgggg

SEQ ID NO: 28 - DNA, 23 bp, Homo sapiens
  source 1..23: mol_type = genomic DNA
ggggccactagggacaggattgg

SEQ ID NO: 29 - DNA, 23 bp, Homo sapiens
  source 1..23: mol_type = genomic DNA
gcatgatgcgcggcccaaggagg

SEQ ID NO: 30 - DNA, 23 bp, Homo sapiens
  source 1..23: mol_type = genomic DNA
gagatgatcgccccttcttctgg

SEQ ID NO: 31 - DNA, 23 bp, Homo sapiens
  source 1..23: mol_type = genomic DNA
gaattactcacgccccaaggagg

SEQ ID NO: 32 - DNA, 72 bp, Homo sapiens
  source 1..72: mol_type = genomic DNA
taatacttttatcttgtcccctccaccccacagtggggccactagggacaggattggtgacagaaaagcccc

SEQ ID NO: 33 - DNA, 70 bp, Homo sapiens
  source 1..70: mol_type = genomic DNA
taatacttttatctgtcaaaaaaaccccacagtggggccactaggacaggattggtgacagaaaagcccc

SEQ ID NO: 34 - DNA, 71 bp, Homo sapiens
  source 1..71: mol_type = genomic DNA
taatacttttatctgtcggggggaccccacagtggggccactagggacaggattggtgacagaaaagcccc

SEQ ID NO: 35 - DNA, 61 bp, Homo sapiens
  source 1..61: mol_type = genomic DNA
taatgcatgatgcgcggcccaaggagggagatgatcgccccttcttctggctctttgagaa

SEQ ID NO: 36 - DNA, 61 bp, Homo sapiens
  source 1..61: mol_type = genomic DNA
taatgaattactcacgccccaaggagggtgatgaccggccgttcttctggatgtttgagaa

SEQ ID NO: 37 - DNA, 110 bp, Homo sapiens
  source 1..110: mol_type = genomic DNA
ggtacttttatctgtcccctccaccccacagtgggccactcgggacaggattggtgacagaaaagccccatccttaggcctcctccttcctagtctcctgatattgggtc

SEQ ID NO: 38 - DNA, 90 bp, Homo sapiens
  source 1..90: mol_type = genomic DNA
ttatctgtcccctccaccccacagtggggccactagggacaggaaaggtgacagaaaagccccatccttaggcctcctccttcctagtct

SEQ ID NO: 39 - skipped

SEQ ID NO: 40 - DNA, 201 bp, Homo sapiens
  variation 27..51: note = n is a, c, g, or t
  variation 93..111: note = n is a, c, g, or t
  variation 152..176: note = n is a, c, g, or t
  source 1..201: mol_type = genomic DNA
tatgaggacgaatctccccgcttatannnnnnnnnnnnnnnnnnnnnnnnnttcttggctttatatatcttgtggaaaggacgaaaacaccgnnnnnnnnnnnnnnnnnnngttttagagctagaaatagcaagttaaaataaggctagtcnnnnnnnnnnnnnnnnnnnnnnnnngtacaagcacacgtttgtcaagacc

SEQ ID NO: 41 - DNA, 415 bp, Homo sapiens
  source 1..415: mol_type = genomic DNA
attcgccctttgtacaaaaaagcaggctttaaaggaaccaattcagtcgactggatccggtaccaaggtcgggcaggaagagggcctatttcccatgattccttcatatttcatatacgatacaaggctgttagagagataataagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttaagttaaaataaggctagtccgttatcaacttgaaaaagtggcaccgagtcggtgctttttttctagacccagctttcttgtacaaagttggcattaaagggcgaat

SEQ ID NO: 42 - DNA, 23 bp, synthetic construct
  misc_feature 1..23: note = reporter oligonucleotide
  source 1..23: mol_type = other DNA
gtcccctccaccccacagtgggg

SEQ ID NO: 43 - DNA, 23 bp, synthetic construct
  misc_feature 1..23: note = reporter oligonucleotide
  source 1..23: mol_type = other DNA
ggggccactagggacaggattgg

SEQ ID NO: 44 - DNA, 100 bp, synthetic construct
  misc_feature 1..100: note = guide RNA
  source 1..100: mol_type = other DNA
  misc_feature 22..25: note = RNA
  misc_feature 31: note = RNA
  misc_feature 37: note = RNA
  misc_feature 44..45: note = RNA
  misc_feature 56: note = RNA
  misc_feature 59: note = RNA
  misc_feature 50: note = RNA
  misc_feature 63..64: note = RNA
  misc_feature 66: note = RNA
  misc_feature 71..72: note = RNA
  misc_feature 94: note = RNA
  misc_feature 97..100: note = RNA
  misc_feature 1..21: note = DNA
  misc_feature 26..30: note = DNA
  misc_feature 32..36: note = DNA
  misc_feature 38..43: note = DNA
  misc_feature 46..49: note = DNA
  misc_feature 51..55: note = DNA
  misc_feature 57..58: note = DNA
  misc_feature 95..96: note = DNA
  misc_feature 60..62: note = DNA
  misc_feature 65: note = DNA
  misc_feature 67..70: note = DNA
  misc_feature 73..93: note = DNA
ggggccactagggacaccatgttttagagctagaaatagcaagttaaaataaggctagtccgttatcaacttcaaaaacacccacccagacggtgctttt

SEQ ID NO: 45 - DNA, 80 bp, synthetic construct
  misc_feature 1..80: note = guide RNA scaffold
  source 1..80: mol_type = other DNA
  misc_feature 2..5: note = RNA
  misc_feature 11: note = RNA
  misc_feature 17: note = RNA
  misc_feature 24..25: note = RNA
  misc_feature 30: note = RNA
  misc_feature 36: note = RNA
  misc_feature 39: note = RNA
  misc_feature 43..44: note = RNA
  misc_feature 46: note = RNA
  misc_feature 51..52: note = RNA
  misc_feature 60: note = RNA
  misc_feature 70: note = RNA
  misc_feature 74: note = RNA
  misc_feature 77..80: note = RNA
  misc_feature 1: note = DNA
  misc_feature 6..10: note = DNA
  misc_feature 12..16: note = DNA
  misc_feature 18..23: note = DNA
  misc_feature 26..29: note = DNA
  misc_feature 31..35: note = DNA
  misc_feature 37..38: note = DNA
  misc_feature 40..42: note = DNA
  misc_feature 45: note = DNA
  misc_feature 47..50: note = DNA
  misc_feature 53..59: note = DNA
  misc_feature 61..69: note = DNA
  misc_feature 71..73: note = DNA
  misc_feature 75..76: note = DNA
gttttagagctagaaatagcaagttaaaataaggctagtccgttatcaacttgaaaaagtggcaccgagtcggtgctttt

SEQ ID NO: 46 - DNA, 76 bp, synthetic construct
  misc_feature 1..76: note = guide RNA scaffold
  source 1..76: mol_type = other DNA
  misc_feature 2..5: note = RNA
  misc_feature 11: note = RNA
  misc_feature 17: note = RNA
  misc_feature 24..25: note = RNA
  misc_feature 30: note = RNA
  misc_feature 36: note = RNA
  misc_feature 39: note = RNA
  misc_feature 43..44: note = RNA
  misc_feature 46: note = RNA
  misc_feature 51..52: note = RNA
  misc_feature 60: note = RNA
  misc_feature 70: note = RNA
  misc_feature 74: note = RNA
  misc_feature 1: note = DNA
  misc_feature 6..10: note = DNA
  misc_feature 12..16: note = DNA
  misc_feature 18..23: note = DNA
  misc_feature 26..29: note = DNA
  misc_feature 31..35: note = DNA
  misc_feature 37..38: note = DNA
  misc_feature 40..42: note = DNA
  misc_feature 45: note = DNA
  misc_feature 47..50: note = DNA
  misc_feature 53..59: note = DNA
  misc_feature 61..69: note = DNA
  misc_feature 71..73: note = DNA
  misc_feature 75..76: note = DNA
gttttagagctagaaatagcaagttaaaataaggctagtccgttatcaacttgaaaaagtggcaccgagtcggtgc

(19) United States
(12) Patent Application Publication
(10) Pub. No.: US 2024/0279677 A1
(43) Pub. Date: Aug. 22, 2024

(54) RNA-Guided Human Genome Engineering

(71) Applicant: President and Fellows of Harvard College, Cambridge, MA (US)

(72) Inventors: George M. Church, Brookline, MA (US); Prashant G. Mali, La Jolla, CA (US); Luhan Yang, Somerville, MA (US)

(21) Appl. No.: 18/619,354

(22) Filed: Mar. 28, 2024

Related U.S. Application Data
(63) Continuation of application No. 17/186,139, filed on Feb. 26, 2021.
Application No. 17/186,139 is a continuation of application No. 16/884,327, filed on May 27, 2020.
Application No. 16/884,327 is a continuation of application No. 16/397,423, filed on Apr. 29, 2019, now Pat. No. 11,359,211.
Application No. 16/397,423 is a continuation of application No. 14/790,147, filed on Jul. 2, 2015, now Pat. No. 10,273,501.
Application No. 14/790,147 is a continuation of application No. 14/653,144, filed on Jun. 17, 2015, now Pat. No. 9,970,024.
(60) Provisional application No. 61/779,169, filed on Mar. 13, 2013.
(60) Provisional application No. 61/738,355, filed on Dec. 17, 2012.

(51) Int. Cl. C12N 15/85; C12N 9/22; C12N 15/01; C12N 15/10; C12N 15/63; C12N 15/81; C12N 15/82; C12N 15/87; C12N 15/90

(52) U.S. Cl. CPC C12N 15/85; C12N 9/22; C12N 15/01; C12N 15/10; C12N 15/102; C12N 15/1024; C12N 15/63; C12N 15/81; C12N 15/8201; C12N 15/87; C12N 15/90; C12N 15/907; C12N 2310/20; C12N 2800/80; C12N 2810/55; C12Y 301/00

(57) ABSTRACT

A method of altering a eukaryotic cell is provided including transfecting the eukaryotic cell with a nucleic acid encoding RNA complementary to genomic DNA of the eukaryotic cell, transfecting the eukaryotic cell with a nucleic acid encoding an enzyme that interacts with the RNA and cleaves the genomic DNA in a site specific manner, wherein the cell expresses the RNA and the enzyme, the RNA binds to complementary genomic DNA and the enzyme cleaves the genomic DNA in a site specific manner.

### RELATED APPLICATION DATA

This application is a continuation application which claims priority to U.S. patent application Ser. No. 17/186,139, filed on Feb. 26, 2021, which is a continuation application which claims priority to U.S. patent application Ser. No. 16/884,327, filed on May 27, 2020, which is a continuation application which claims priority to U.S. patent application Ser. No. 16/397,423, now U.S. Pat. No. 11,359,211, filed on Apr. 29, 2019, which is a continuation application which claims priority to U.S. patent application Ser. No. 14/790,147, now U.S. Pat. No. 10,273,501, filed on Jul. 2, 2015, which is a continuation application which claims priority to U.S. patent application Ser. No. 14/653,144, now U.S. Pat. No. 9,970,024, filed on Jun. 17, 2015, which is a National Stage Application under 35 U.S.C. 371 of co-pending PCT application PCT/US2013/075317, filed Dec. 16, 2013 and U.S. provisional application No. 61/799,169, filed Mar. 13, 2013 and U.S. provisional application No. 61/738,355, filed Dec. 17, 2012 each of which is hereby incorporated herein by reference in their entirety for all purposes.

### STATEMENT OF GOVERNMENT INTERESTS

This invention was made with government support under P50 HG005550 awarded by National Institutes of Health. The government has certain rights in the invention.

### BACKGROUND

Bacterial and archaeal CRISPR systems rely on crRNAs in complex with Cas proteins to direct degradation of complementary sequences present within invading viral and plasmid DNA (1-3). A recent in vitro reconstitution of the S. pyogenes type II CRISPR system demonstrated that crRNA fused to a normally trans-encoded tracrRNA is sufficient to direct Cas9 protein to sequence-specifically cleave target DNA sequences matching the crRNA (4).

### SEQUENCE LISTING

The instant application contains a Sequence Listing which has been submitted electronically in XML format and is hereby incorporated by reference in its entirety. Said XML copy, created on Mar. 27, 2024, is named “Sequence_Listing_010498.01584_ST26” and is 71 KB in size.

### SUMMARY

The present disclosure references documents numerically which are listed at the end of the present disclosure. The document corresponding to the number is incorporated by reference into the specification as a supporting reference corresponding to the number as if fully cited.

According to one aspect of the present disclosure, a eukaryotic cell is transfected with a two component system including RNA complementary to genomic DNA and an enzyme that interacts with the RNA. The RNA and the enzyme are expressed by the cell. The RNA of the RNA/enzyme complex then binds to complementary genomic DNA. The enzyme then performs a function, such as cleavage of the genomic DNA. The RNA includes between about 10 nucleotides to about 250 nucleotides. The RNA includes between about 20 nucleotides to about 100 nucleotides. According to certain aspects, the enzyme may perform any desired function in a site specific manner for which the enzyme has been engineered. According to one aspect, the eukaryotic cell is a yeast cell, plant cell or mammalian cell. According to one aspect, the enzyme cleaves genomic sequences targeted by RNA sequences (see references (4-6)), thereby creating a genomically altered eukaryotic cell.

According to one aspect, the present disclosure provides a method of genetically altering a human cell by including a nucleic acid encoding an RNA complementary to genomic DNA into the genome of the cell and a nucleic acid encoding an enzyme that performs a desired function on genomic DNA into the genome of the cell. According to one aspect, the RNA and the enzyme are expressed. According to one aspect, the RNA hybridizes with complementary genomic DNA. According to one aspect, the enzyme is activated to perform a desired function, such as cleavage, in a site specific manner when the RNA is hybridized to the complementary genomic DNA. According to one aspect, the RNA and the enzyme are components of a bacterial Type II CRISPR system.

According to one aspect, a method of altering a eukaryotic cell is providing including transfecting the eukaryotic cell with a nucleic acid encoding RNA complementary to genomic DNA of the eukaryotic cell, transfecting the eukaryotic cell with a nucleic acid encoding an enzyme that interacts with the RNA and cleaves the genomic DNA in a site specific manner, wherein the cell expresses the RNA and the enzyme, the RNA binds to complementary genomic DNA and the enzyme cleaves the genomic DNA in a site specific manner. According to one aspect, the enzyme is Cas9 or modified Cas9 or a homolog of Cas9. According to one aspect, the eukaryotic cell is a yeast cell, a plant cell or a mammalian cell. According to one aspect, the RNA includes between about 10 to about 250 nucleotides. According to one aspect, the RNA includes between about 20 to about 100 nucleotides.

According to one aspect, a method of altering a human cell is provided including transfecting the human cell with a nucleic acid encoding RNA complementary to genomic DNA of the eukaryotic cell, transfecting the human cell with a nucleic acid encoding an enzyme that interacts with the RNA and cleaves the genomic DNA in a site specific manner, wherein the human cell expresses the RNA and the enzyme, the RNA binds to complementary genomic DNA and the enzyme cleaves the genomic DNA in a site specific manner. According to one aspect, the enzyme is Cas9 or modified Cas9 or a homolog of Cas9. According to one aspect, the RNA includes between about 10 to about 250 nucleotides. According to one aspect, the RNA includes between about 20 to about 100 nucleotides.

According to one aspect, a method of altering a eukaryotic cell at a plurality of genomic DNA sites is provided including transfecting the eukaryotic cell with a plurality of nucleic acids encoding RNAs complementary to different sites on genomic DNA of the eukaryotic cell, transfecting the eukaryotic cell with a nucleic acid encoding an enzyme that interacts with the RNA and cleaves the genomic DNA in a site specific manner, wherein the cell expresses the RNAs and the enzyme, the RNAs bind to complementary genomic DNA and the enzyme cleaves the genomic DNA in a site specific manner. According to one aspect, the enzyme is Cas9. According to one aspect, the eukaryotic cell is a yeast cell, a plant cell or a mammalian cell. According to one aspect, the RNA includes between about 10 to about 250 nucleotides. According to one aspect, the RNA includes between about 20 to about 100 nucleotides.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1A depicts genome editing in human cells using an engineered type II CRISPR system (SEQ ID NO:17 and 45-46). FIG. 1B depicts a genomically integrated GFP coding sequence disrupted by the insertion of a stop codon and a 68 bp genomic fragment from the AAVS1 locus (SEQ ID NO:18). FIG. 1C-1 bar graph depicts HR efficiencies induced by T1, T2, and TALEN-mediated nuclease activity at the target locus, as measured by FACS. FIG. 1C-2 depicts FACS plots and microscopy images of the targeted cells.

FIG. 2A depicts RNA-guided genome editing of the native AAVS1 locus in multiple cell types (SEQ ID NO:19). FIG. 2B-1, FIG. 2B-2, and FIG. 2B-3 depict total count and location of deletions caused by NHEJ in 293 Ts, K562s, and PGP1 iPS cells. FIG. 2C depicts DNA donor architecture for HR at the AAVS1 locus, and the locations of sequencing primers (arrows) for detecting successful targeted events. FIG. 2D depicts PCR assay three days post transfection. FIG. 2E depicts successful HR confirmed by Sanger sequencing of the PCR amplicon (SEQ ID NOs:20-21). FIG. 2F depicts images of targeted clones of 293T cells.

FIG. 3A-1 and FIG. 3A-2 depict a human codon-optimized version of the Cas9 protein and full sequence of the cas9 gene insert (SEQ ID NO:22). FIG. 3B depicts U6 promoter based expression scheme for the guide RNAs and predicted RNA transcript secondary structure (SEQ ID NO:23). FIG. 3C depicts targeted GN20GG genomic sites and selected gRNAs (SEQ ID NOs:24-31).

FIG. 4 depicts that all possible combinations of the repair DNA donor, Cas9 protein, and gRNA were tested for their ability to effect successful HR in 293 Ts.

FIG. 5A-5B depict the analysis of gRNA and Cas9 mediated genome editing (SEQ ID NO:19).

FIGS. 6A-6B depict 293T stable lines each bearing a distinct GFP reporter construct (SEQ ID NOs:32-34).

FIG. 7 depicts gRNAs targeting the flanking GFP sequences of the reporter described in FIG. 1B (in 293 Ts).

FIGS. 8A-8B depict 293T stable lines each bearing a distinct GFP reporter construct (SEQ ID NOs:35-36).

FIGS. 9A-9C depict human iPS cells (PGP1) that were nucleofected with constructs (SEQ ID NO:19).

FIGS. 10A-10B depict RNA-guided NHEJ in K562 cells (SEQ ID NO:19).

FIG. 11A-11B depict RNA-guided NHEJ in 293T cells (SEQ ID NO:19).

FIGS. 12A-12C depict HR at the endogenous AAVS1 locus using either a dsDNA donor or a short oligonucleotide donor (SEQ ID NOs:37-38).

FIG. 13A-1 and FIG. 13A-2 and FIG. 13B depict the methodology for multiplex synthesis, retrieval and U6 expression vector cloning of guide RNAs targeting genes in the human genome (SEQ ID NOs:39-41).

FIGS. 14A-14D depict CRISPR mediated RNA-guided transcriptional activation (SEQ ID NOs:42-43).

FIGS. 15A-15B depict gRNA sequence flexibility and applications thereof (SEQ ID NO:44-46).

### DETAILED DESCRIPTION

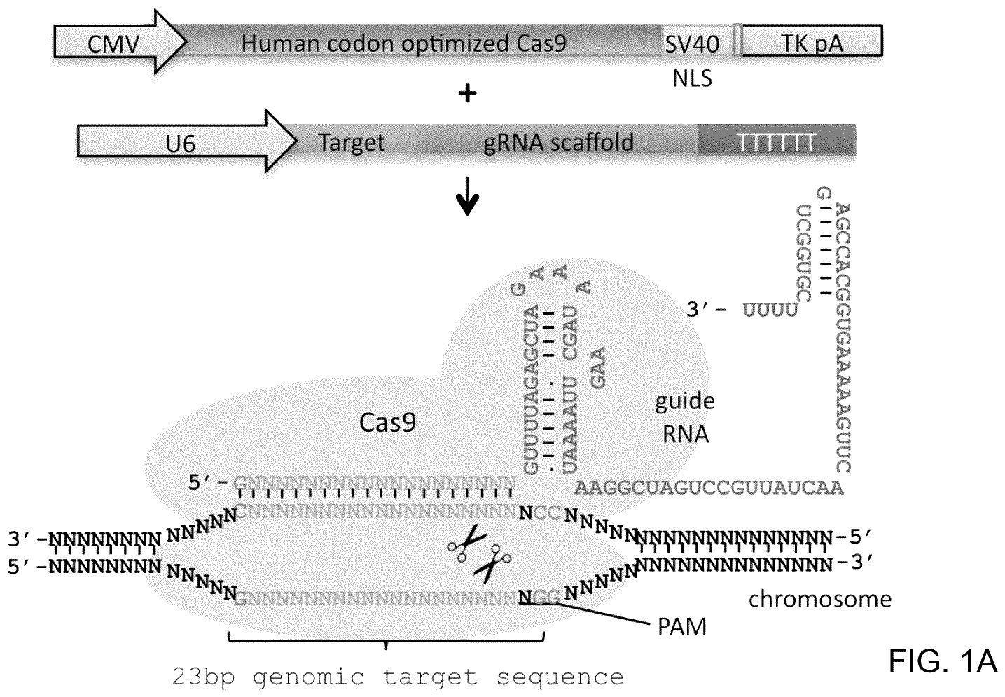

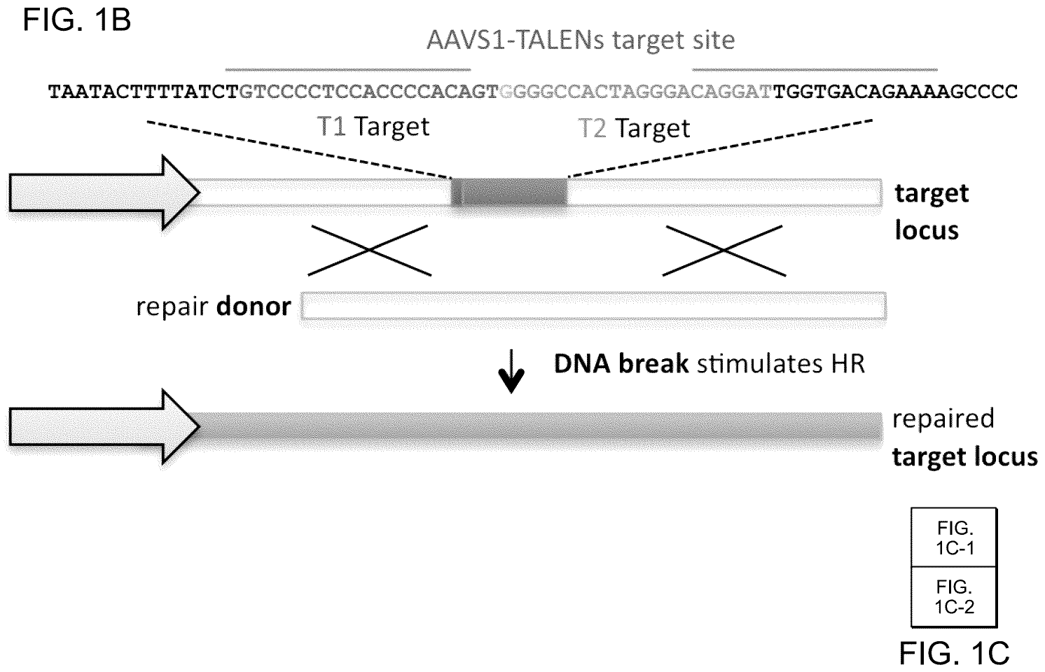

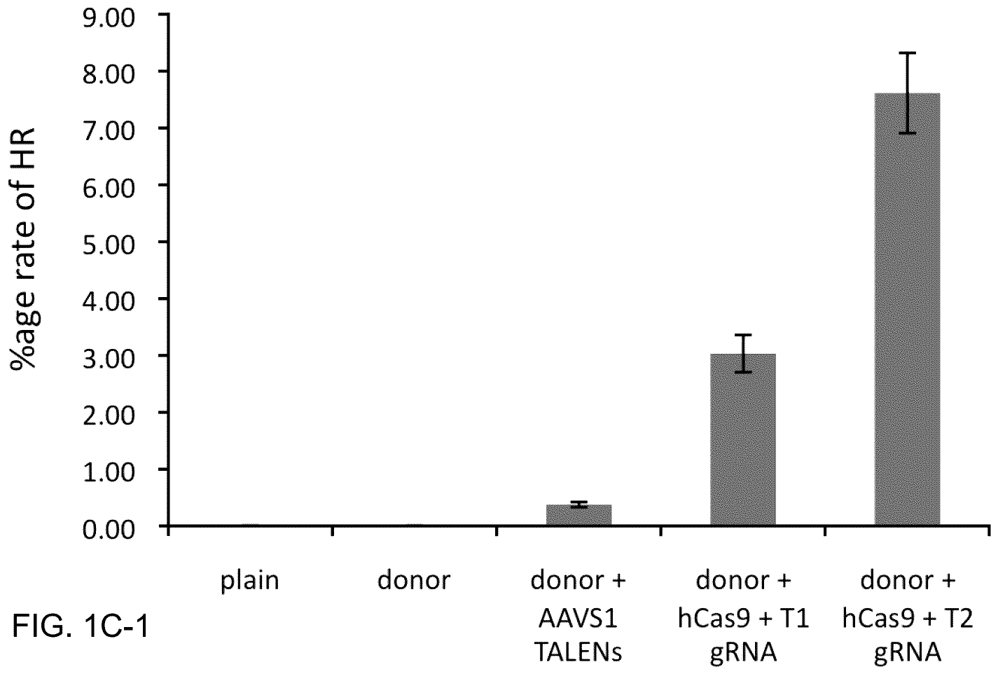

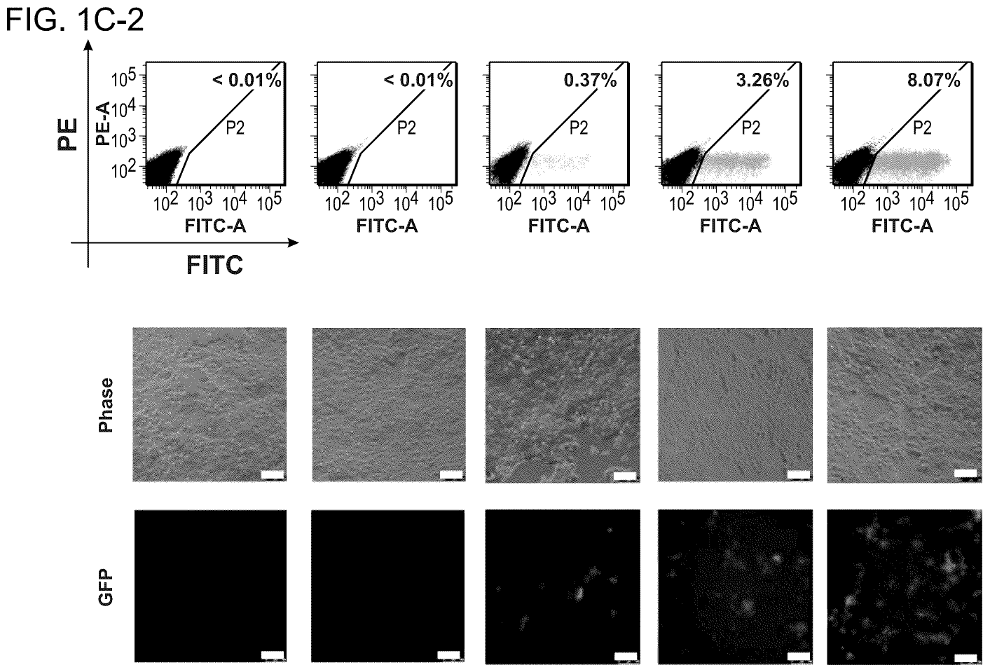

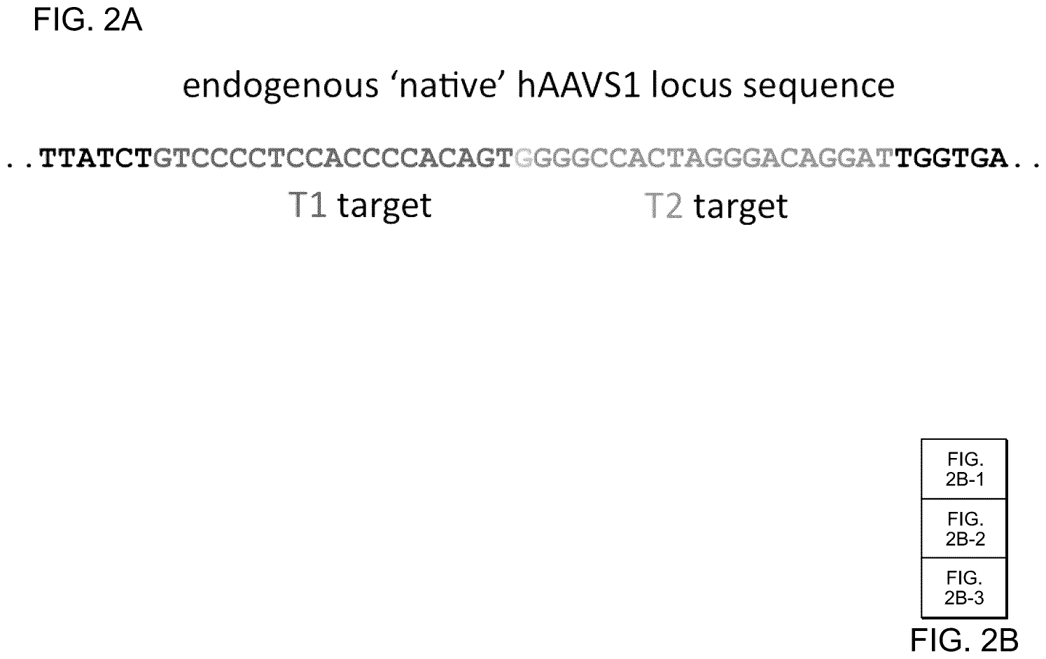

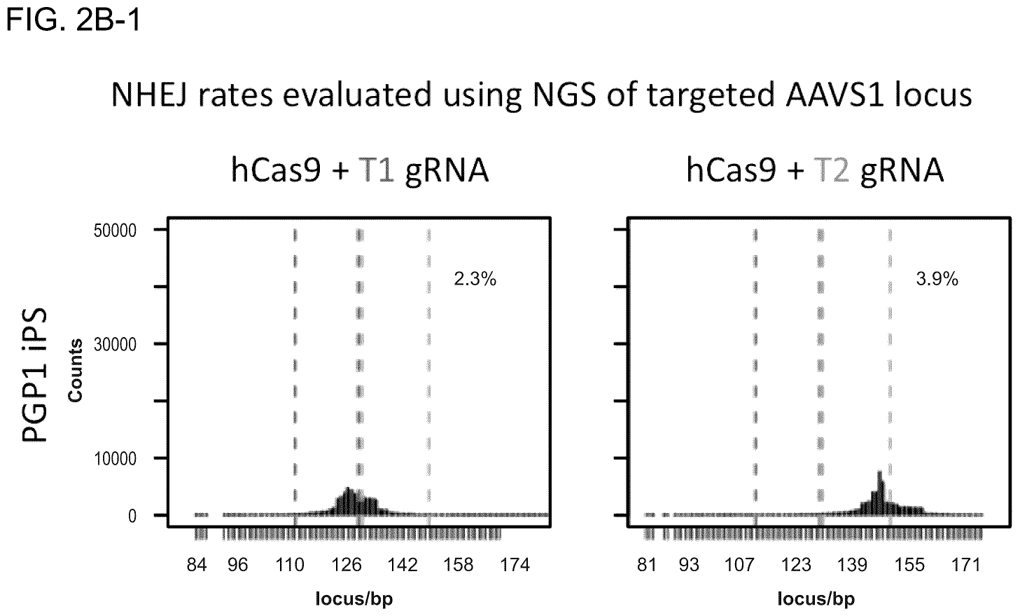

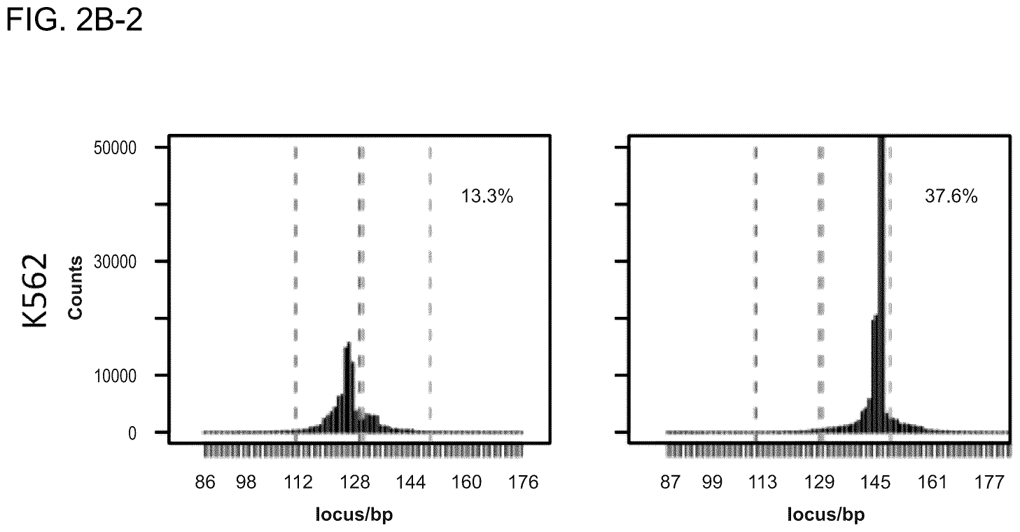

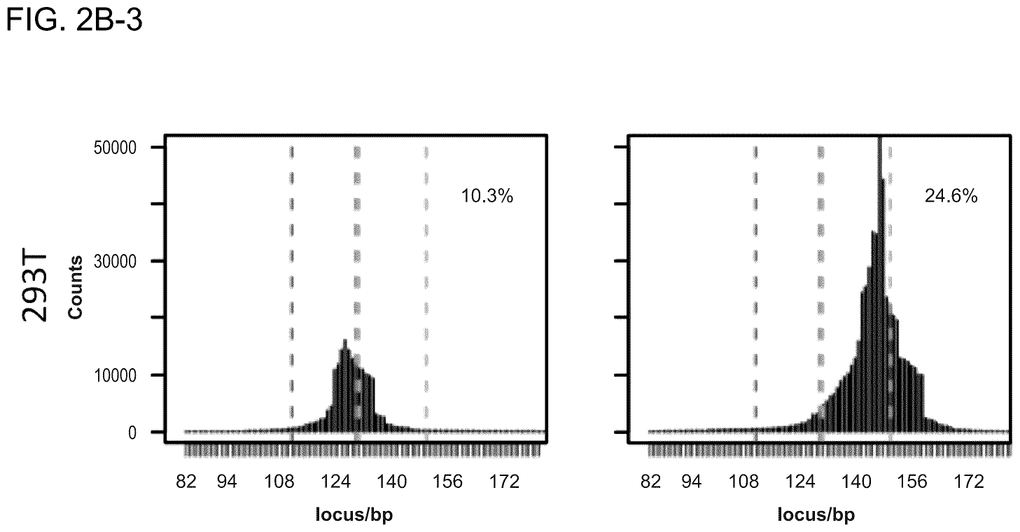

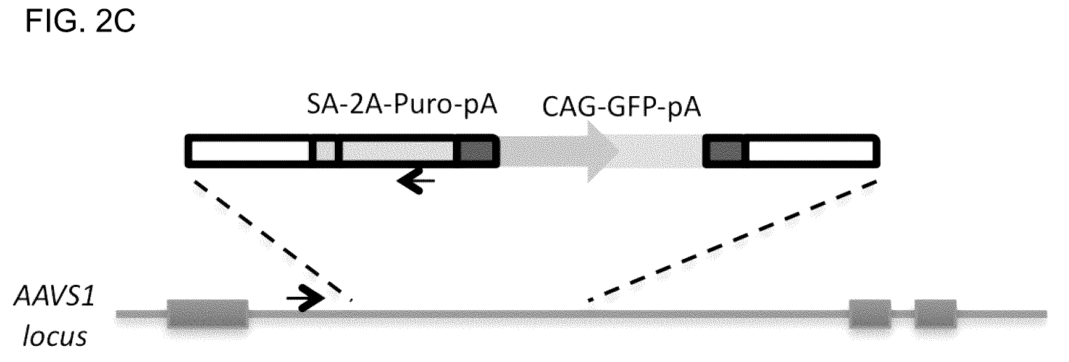

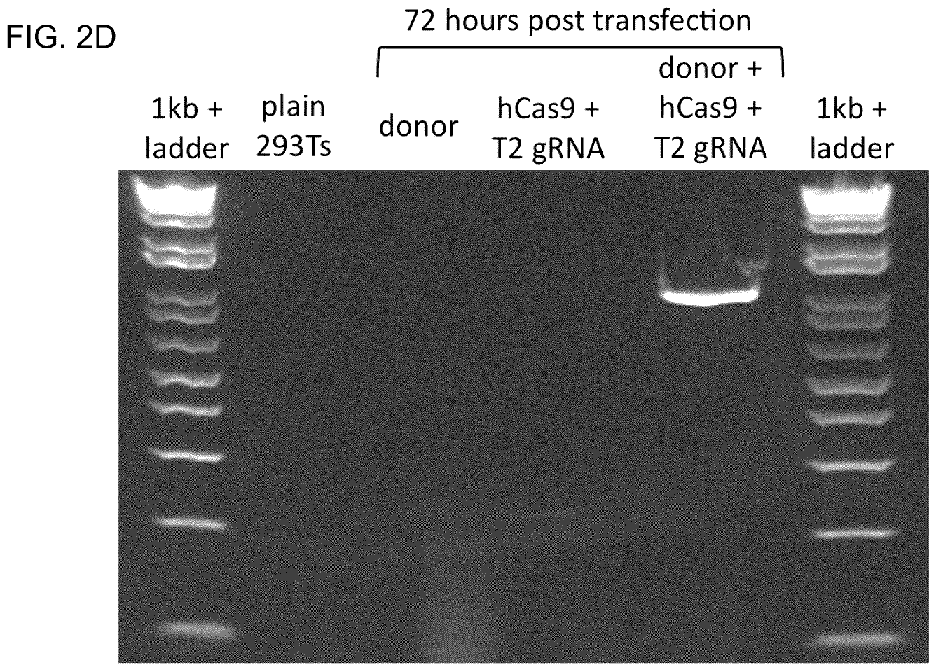

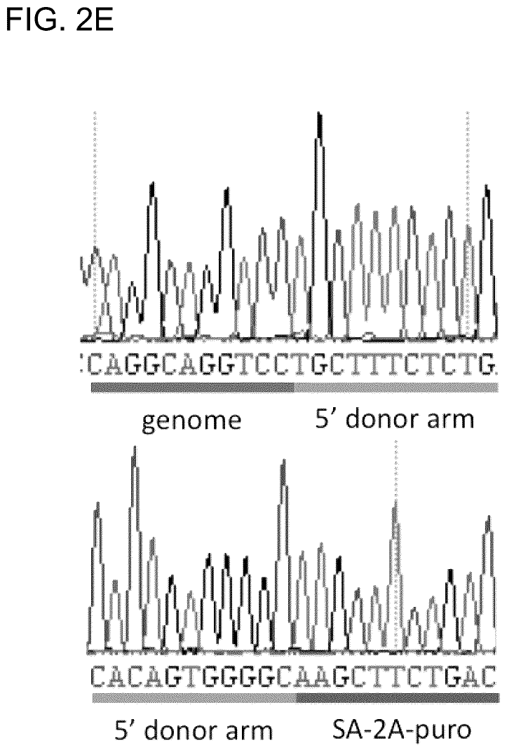

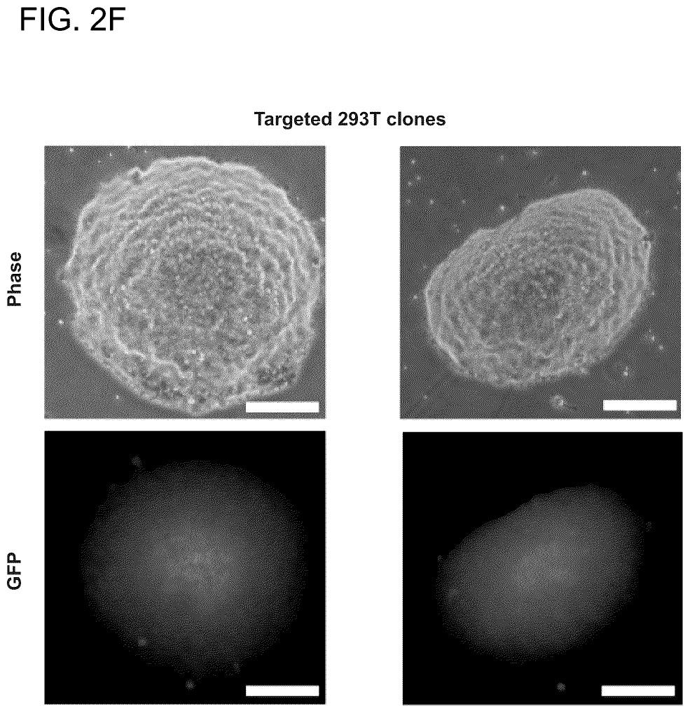

According to one aspect, a human codon-optimized version of the Cas9 protein bearing a C-terminus SV40 nuclear localization signal is synthetized and cloned into a mammalian expression system (FIG. 1A and FIGS. 3A-1 and 3A-2). Accordingly, FIG. 1 is directed to genome editing in human cells using an engineered type II CRISPR system. As shown in FIG. 1A, RNA-guided gene targeting in human cells involves co-expression of the Cas9 protein bearing a C-terminus SV40 nuclear localization signal with one or more guide RNAs (gRNAs) expressed from the human U6 polymerase III promoter. Cas9 unwinds the DNA duplex and cleaves both strands upon recognition of a target sequence by the gRNA, but only if the correct protospacer-adjacent motif (PAM) is present at the 3′ end. Any genomic sequence of the form GN20GG can in principle be targeted. As shown in FIG. 1B, a genomically integrated GFP coding sequence is disrupted by the insertion of a stop codon and a 68 bp genomic fragment from the AAVS1 locus. Restoration of the GFP sequence by homologous recombination (HR) with an appropriate donor sequence results in GFP⁺ cells that can be quantitated by FACS. T1 and T2 gRNAs target sequences within the AAVS1 fragment. Binding sites for the two halves of the TAL effector nuclease heterodimer (TALEN) are underlined. As shown in FIG. 1C-1, bar graph depict HR efficiencies induced by T1, T2, and TALEN-mediated nuclease activity at the target locus, as measured by FACS. Representative FACS plots and microscopy images of the targeted cells, FIG. 1C-2, are depicted below (scale bar is 100 microns). Data is mean+/−SEM (N=3).

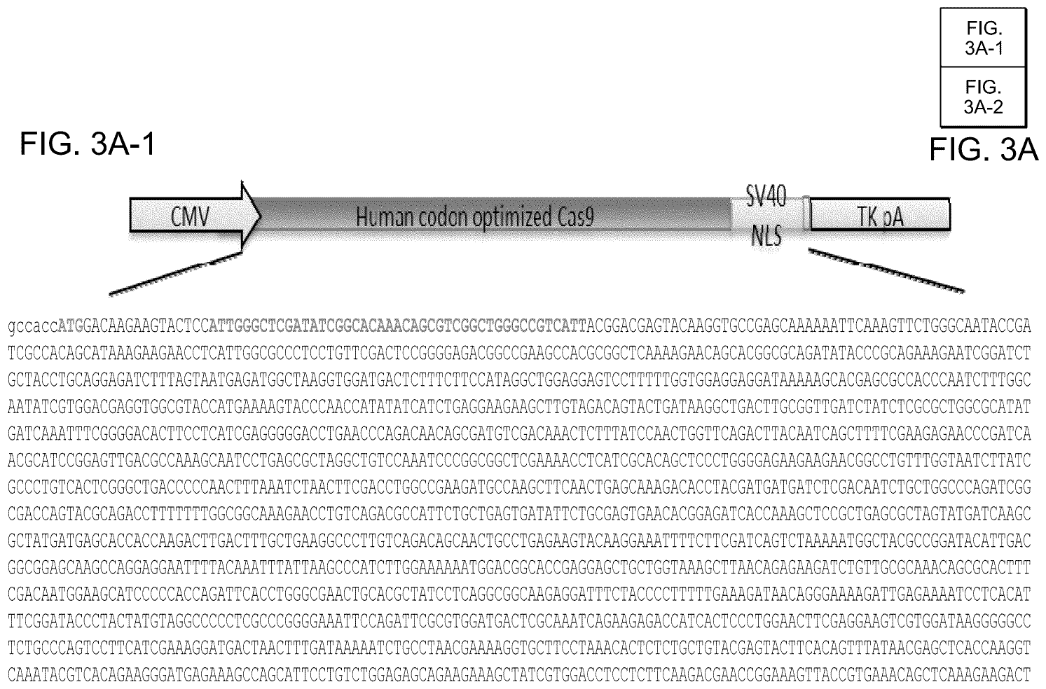

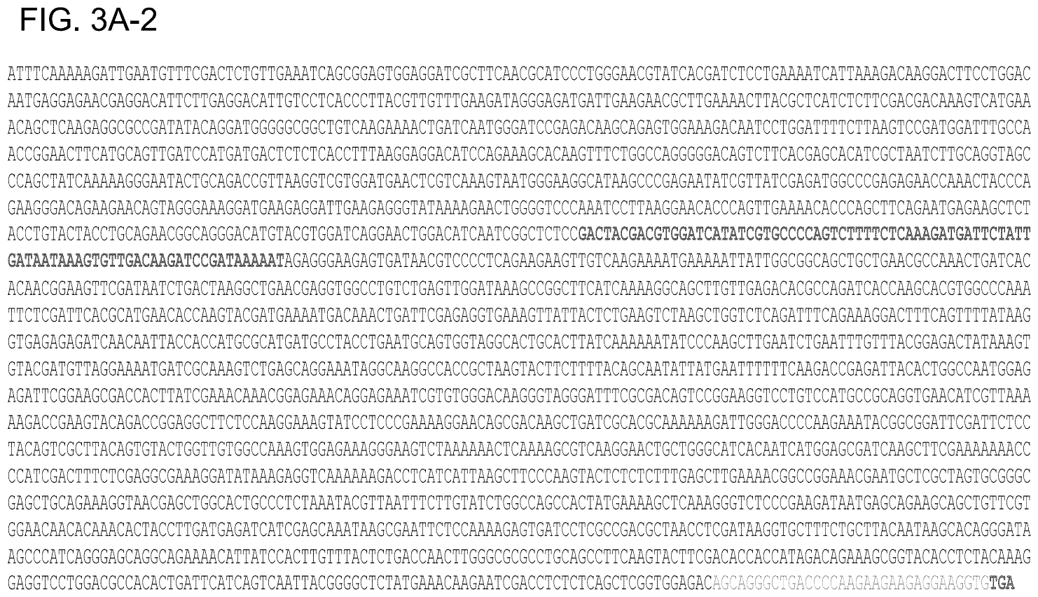

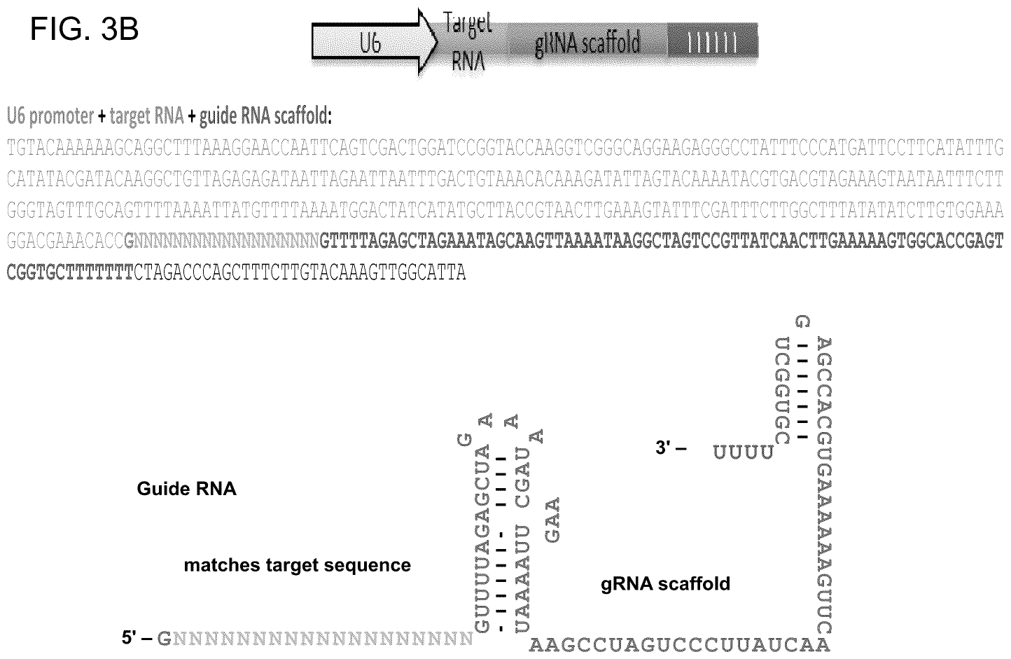

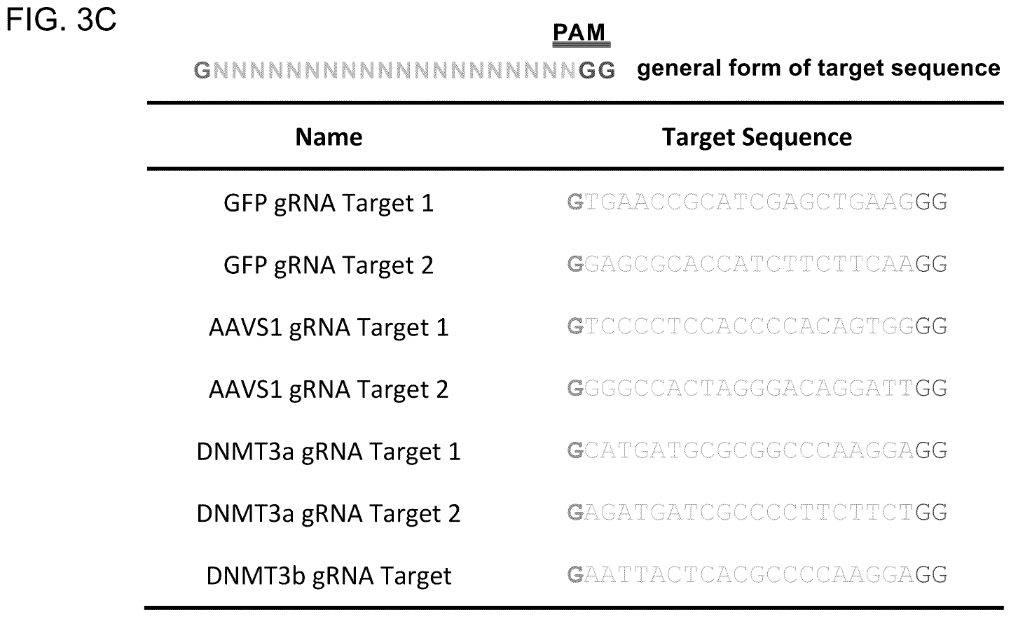

According to one aspect, to direct Cas9 to cleave sequences of interest, crRNA-tracrRNA fusion transcripts are expressed, hereafter referred to as guide RNAs (gRNAs), from the human U6 polymerase III promoter. According to one aspect, gRNAs are directly transcribed by the cell. This aspect advantageously avoids reconstituting the RNA processing machinery employed by bacterial CRISPR systems (FIG. 1A and FIG. 3B) (see references (4, 7-9)). According to one aspect, a method is provided for altering genomic DNA using a U6 transcription initiating with G and a PAM (protospacer-adjacent motif) sequence-NGG following the 20 bp crRNA target. According to this aspect, the target genomic site is in the form of GN20GG (See FIG. 3C).

According to one aspect, a GFP reporter assay (FIG. 1B) in 293T cells was developed similar to one previously described (see reference (10)) to test the functionality of the genome engineering methods described herein. According to one aspect, a stable cell line was established bearing a genomically integrated GFP coding sequence disrupted by the insertion of a stop codon and a 68 bp genomic fragment from the AAVS1 locus that renders the expressed protein fragment non-fluorescent. Homologous recombination (HR) using an appropriate repair donor can restore the normal GFP sequence, which allows one to quantify the resulting GFP⁺ cells by flow activated cell sorting (FACS).

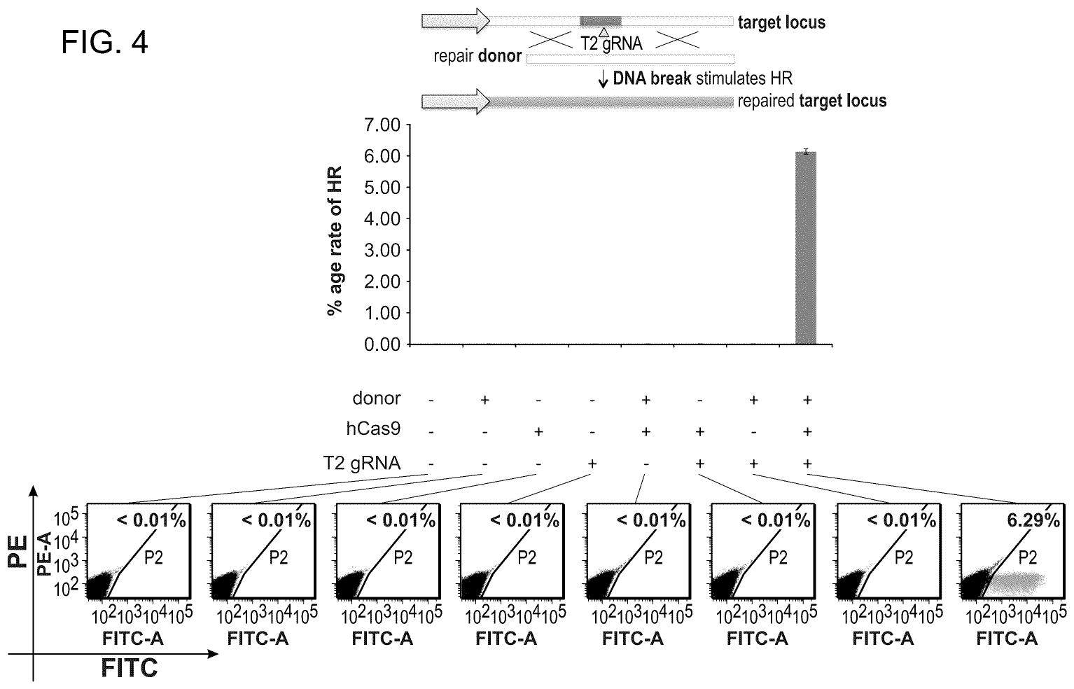

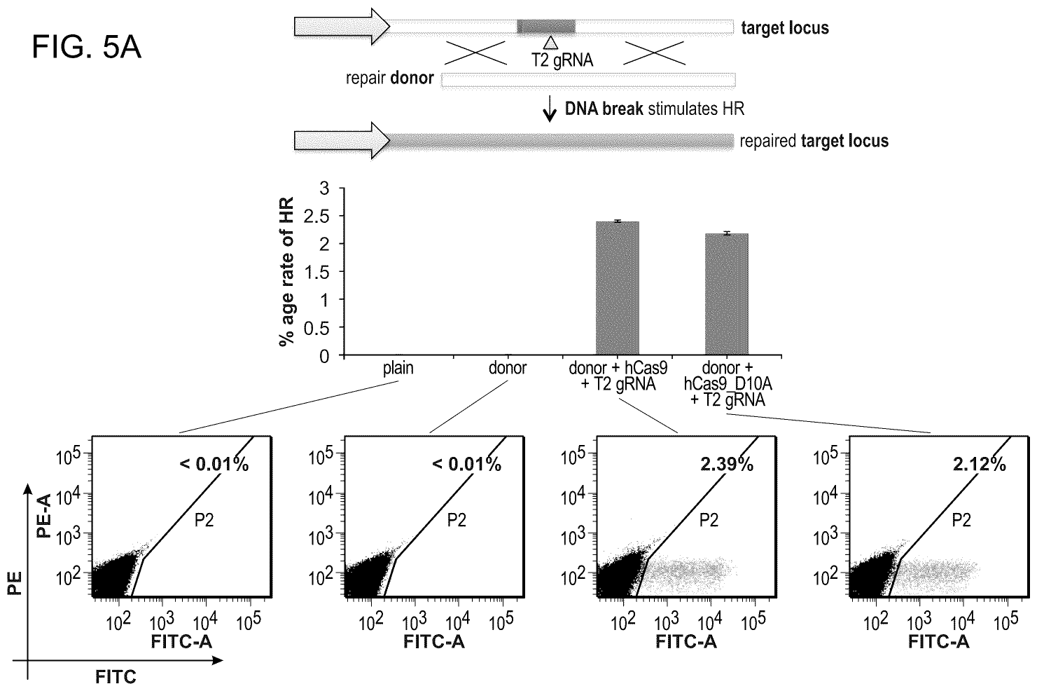

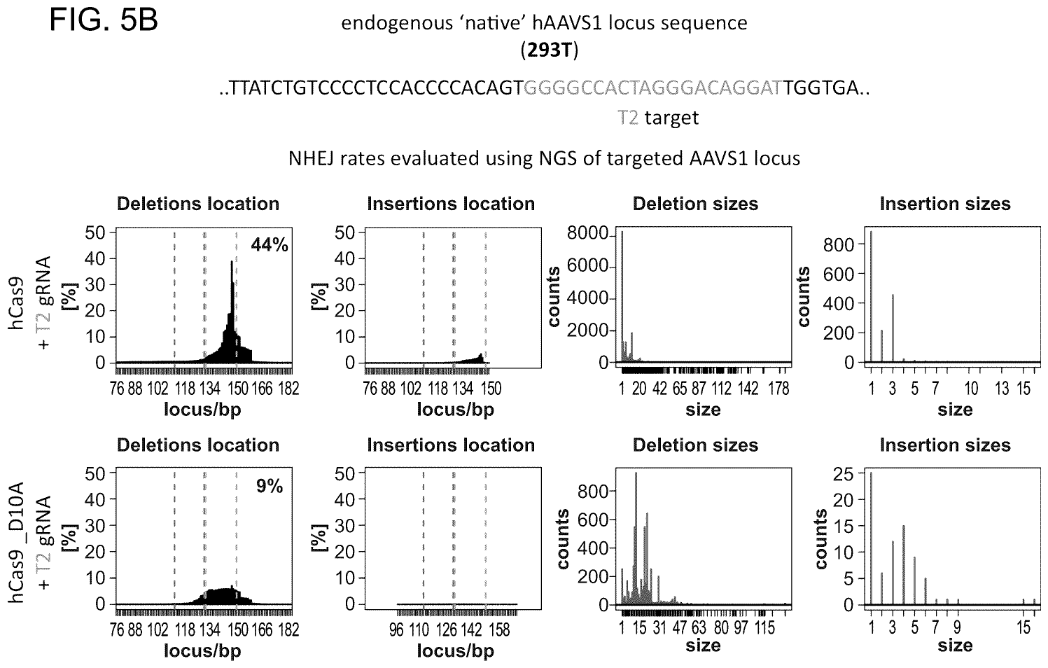

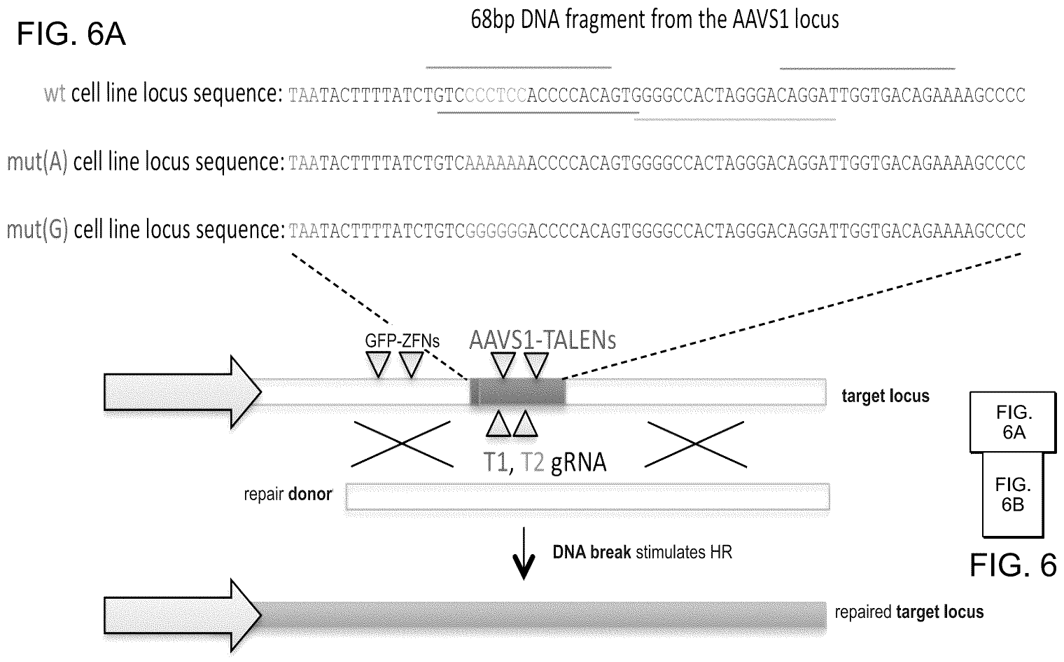

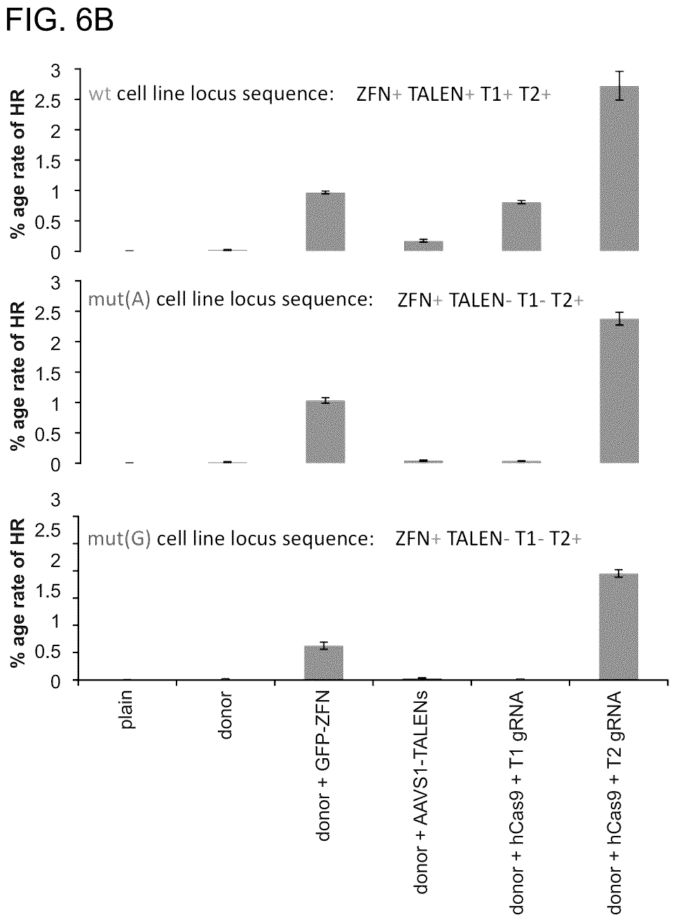

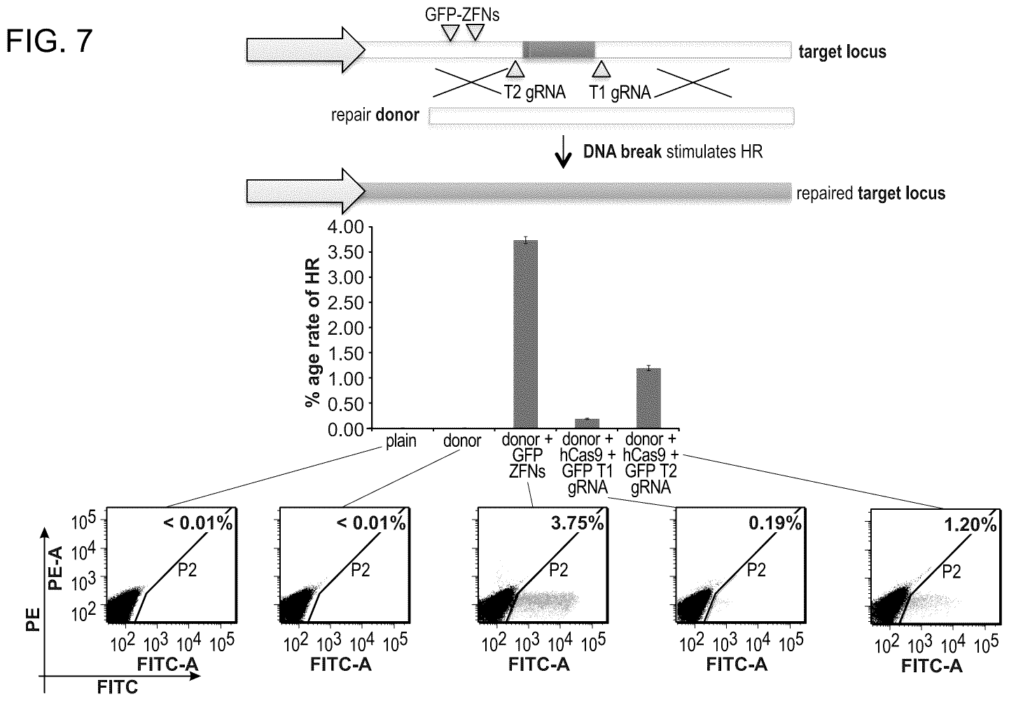

According to one aspect, a method is provided of homologous recombination (HR). Two gRNAs are constructed, T1 and T2, that target the intervening AAVS1 fragment (FIG. 1b). Their activity to that of a previously described TAL effector nuclease heterodimer (TALEN) targeting the same region (see reference (11)) was compared. Successful HR events were observed using all three targeting reagents, with gene correction rates using the T1 and T2 gRNAs approaching 3% and 8% respectively (FIG. 1C-1). This RNA-mediated editing process was notably rapid, with the first detectable GFP⁺ cells appearing ˜20 hours post transfection compared to ˜40 hours for the AAVS1 TALENs. HR was observed only upon simultaneous introduction of the repair donor, Cas9 protein, and gRNA, confirming that all components are required for genome editing (FIG. 4). While no apparent toxicity associated with Cas9/crRNA expression was noted, work with ZFNs and TALENs has shown that nicking only one strand further reduces toxicity. Accordingly, a Cas9D10A mutant was tested that is known to function as a nickase in vitro, which yielded similar HR but lower non-homologous end joining (NHEJ) rates (FIG. 5) (see references (4, 5)). Consistent with (4) where a related Cas9 protein is shown to cut both strands 6 bp upstream of the PAM, NHEJ data confirmed that most deletions or insertions occurred at the 3′ end of the target sequence (FIG. 5B). Also confirmed was that mutating the target genomic site prevents the gRNA from effecting HR at that locus, demonstrating that CRISPR-mediated genome editing is sequence specific (FIGS. 6A and 6B). It was showed that two gRNAs targeting sites in the GFP gene, and also three additional gRNAs targeting fragments from homologous regions of the DNA methyl transferase 3a (DNMT3a) and DNMT3b genes could sequence specifically induce significant HR in the engineered reporter cell lines (FIG. 7, 8). Together these results confirm that RNA-guided genome targeting in human cells induces robust HR across multiple target sites.

According to certain aspects, a native locus was modified. gRNAs were used to target the AAVS1 locus located in the PPP1R12C gene on chromosome 19, which is ubiquitously expressed across most tissues (FIG. 2A) in 293 Ts, K562s, and PGP1 human iPS cells (see reference (12)) and analyzed the results by next-generation sequencing of the targeted locus. Accordingly, FIG. 2 is directed to RNA-guided genome editing of the native AAVS1 locus in multiple cell types. As shown in FIG. 2A, T1 (red) and T2 (green) gRNAs target sequences in an intron of the PPP1R12C gene within the chromosome 19 AAVS1 locus. As shown in FIGS. 2B-1, 2B-2, and 2B-3, total count and location of deletions caused by NHEJ in 293 Ts, K562s, and PGP1 iPS cells following expression of Cas9 and either T1 or T2 gRNAs as quantified by next-generation sequencing is provided. Red and green dash lines demarcate the boundaries of the T1 and T2 gRNA targeting sites. NHEJ frequencies for T1 and T2 gRNAs were 10% and 25% in 293T, 13% and 38% in K562, and 2% and 4% in PGP1 iPS cells, respectively. As shown in FIG. 2C, DNA donor architecture for HR at the AAVS1 locus, and the locations of sequencing primers (arrows) for detecting successful targeted events, are depicted. As shown in FIG. 2D, PCR assay three days post transfection demonstrates that only cells expressing the donor, Cas9 and T2 gRNA exhibit successful HR events. As shown in FIG. 2E, successful HR was confirmed by Sanger sequencing of the PCR amplicon showing that the expected DNA bases at both the genome-donor and donor-insert boundaries are present. As shown in FIG. 2F, successfully targeted clones of 293T cells were selected with puromycin for 2 weeks. Microscope images of two representative GFP+ clones is shown (scale bar is 100 microns).

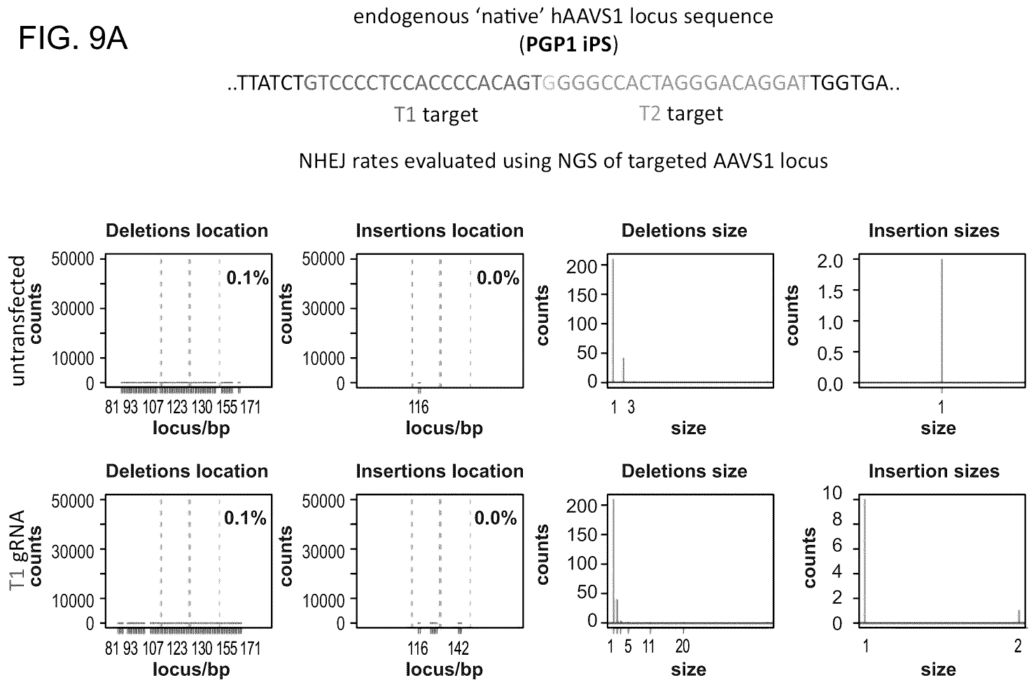

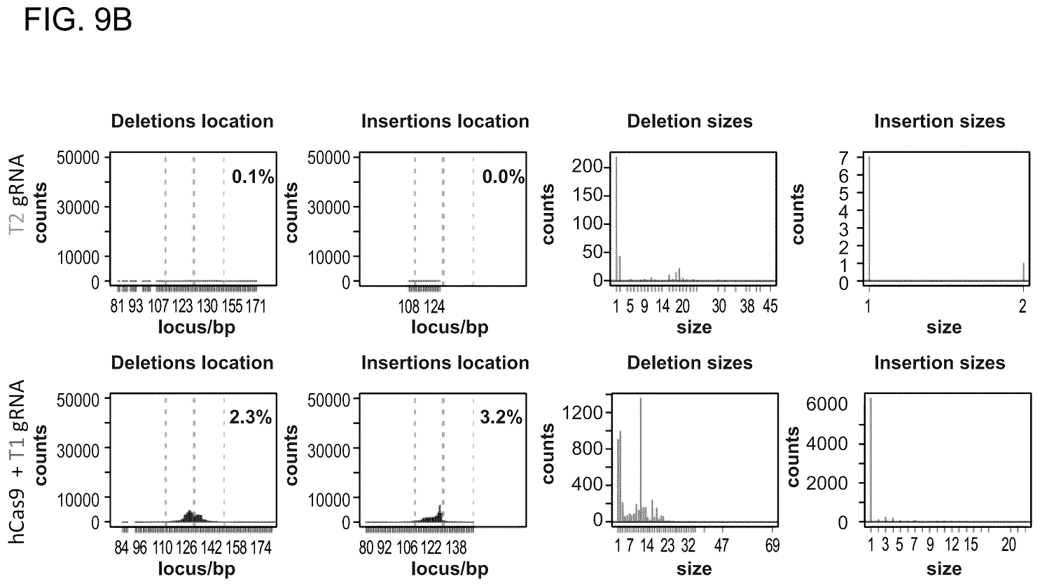

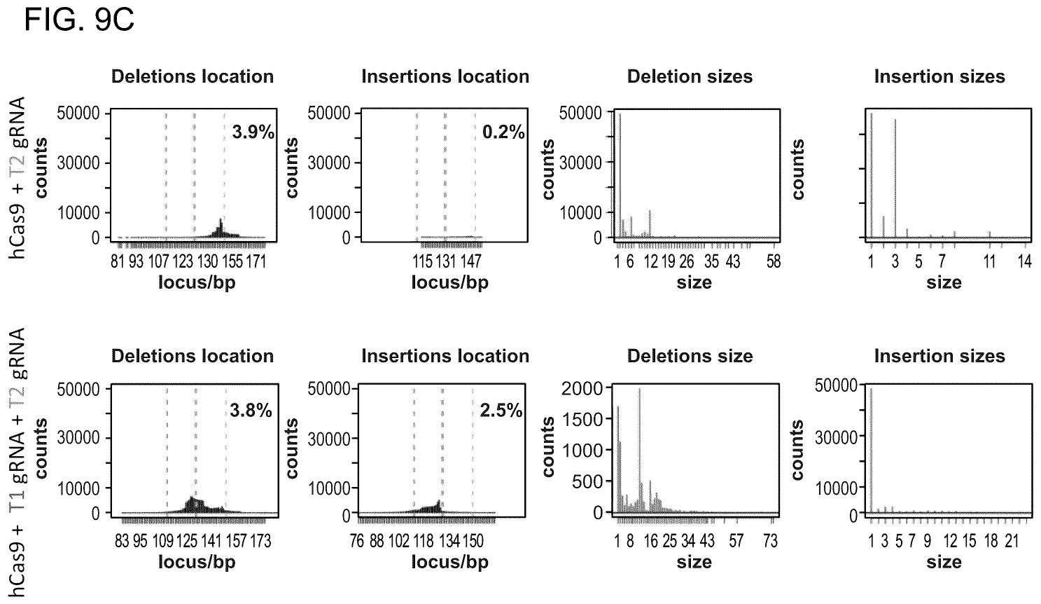

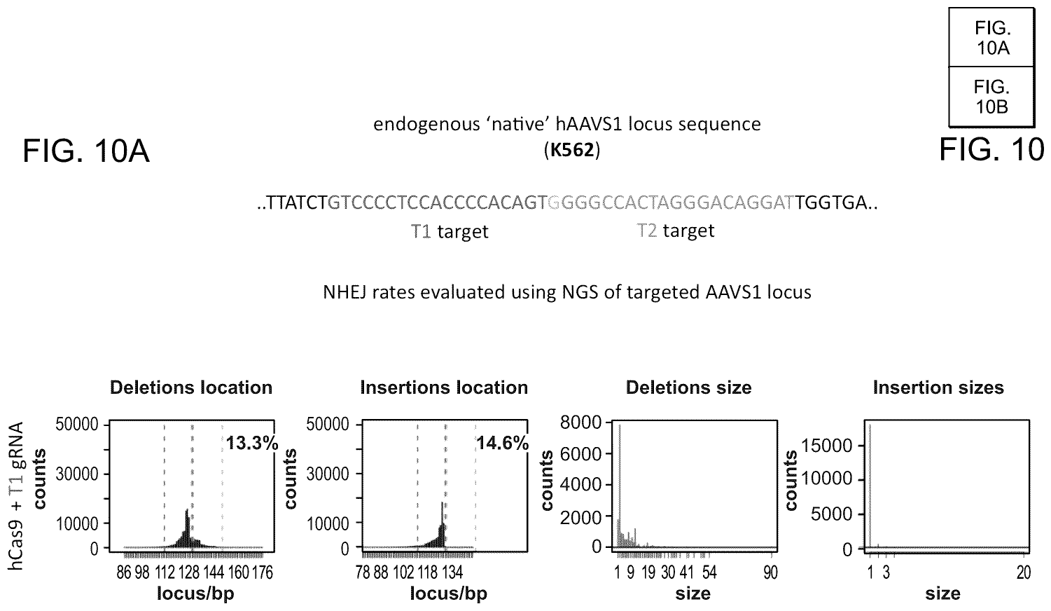

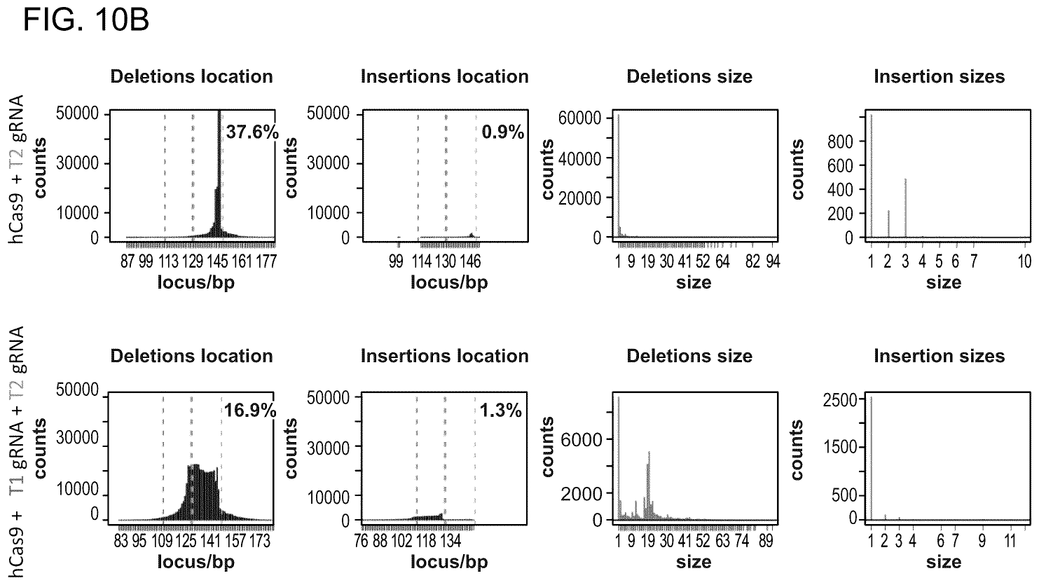

Consistent with results for the GFP reporter assay, high numbers of NHEJ events were observed at the endogenous locus for all three cell types. The two gRNAs T1 and T2 achieved NHEJ rates of 10 and 25% in 293 Ts, 13 and 38% in K562s, and 2 and 4% in PGP1-iPS cells, respectively (FIGS. 2B-1, 2B-2, and 2B-3). No overt toxicity was observed from the Cas9 and crRNA expression required to induce NHEJ in any of these cell types (FIGS. 9A, 9B, and 9C). As expected, NHEJ-mediated deletions for T1 and T2 were centered around the target site positions, further validating the sequence specificity of this targeting process (FIGS. 9A-C, 10, 11). Simultaneous introduction of both T1 and T2 gRNAs resulted in high efficiency deletion of the intervening 19 bp fragment (FIGS. 10A and 10B), demonstrating that multiplexed editing of genomic loci is feasible using this approach.

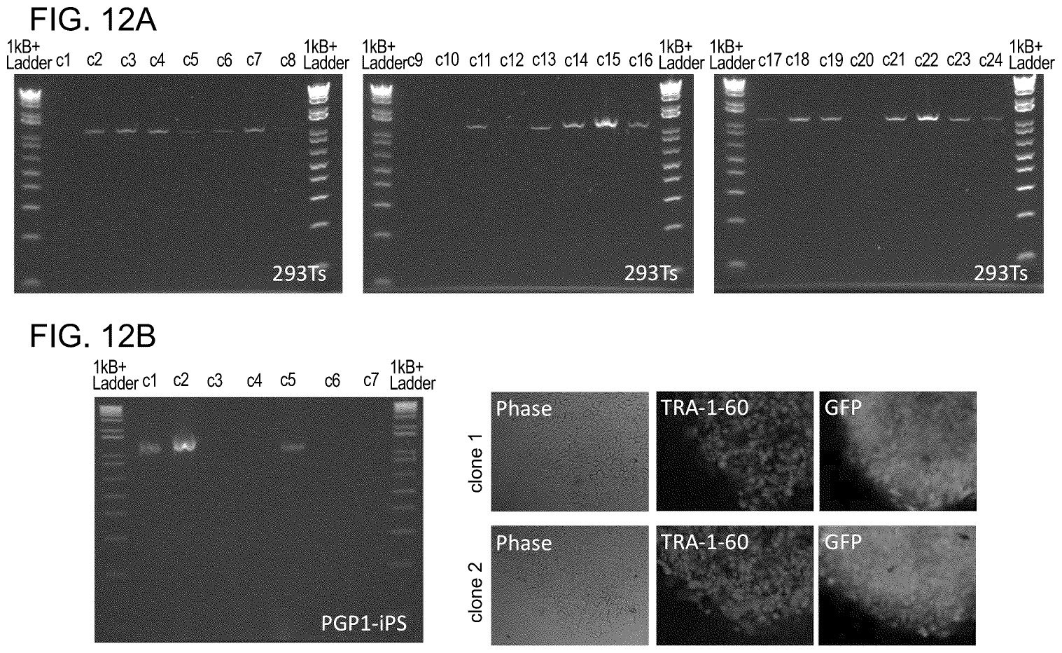

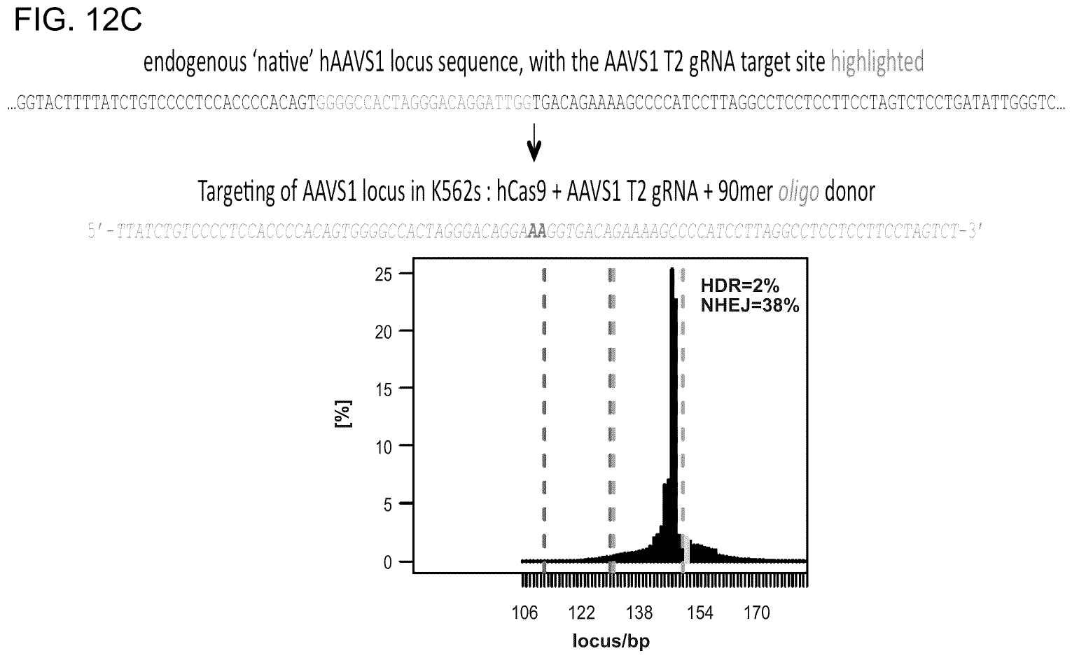

According to one aspect, HR is used to integrate either a dsDNA donor construct (see reference (13)) or an oligo donor into the native AAVS1 locus (FIG. 2C, FIG. 12C). HR-mediated integration was confirmed using both approaches by PCR (FIG. 2D, FIG. 12A) and Sanger sequencing (FIG. 2E). 293T or iPS clones were readily derived from the pool of modified cells using puromycin selection over two weeks (FIG. 2F, FIG. 12B). These results demonstrate that Cas9 is capable of efficiently integrating foreign DNA at endogenous loci in human cells. Accordingly, one aspect of the present disclosure includes a method of integrating foreign DNA into the genome of a cell using homologous recombination and Cas9.

According to one aspect, an RNA-guided genome editing system is provided which can readily be adapted to modify other genomic sites by simply modifying the sequence of the gRNA expression vector to match a compatible sequence in the locus of interest. According to this aspect, 190,000 specifically gRNA-targetable sequences targeting about 40.5% exons of genes in the human genome were generated. These target sequences were incorporated into a 200 bp format compatible with multiplex synthesis on DNA arrays (see reference (14)) (FIGS. 13A-1 and 13A-2). According to this aspect, a ready genome-wide reference of potential target sites in the human genome and a methodology for multiplex gRNA synthesis is provided.

According to one aspect, methods are provided for multiplexing genomic alterations in a cell by using one or more or a plurality of RNA/enzyme systems described herein to alter the genome of a cell at a plurality of locations. According to one aspect, target sites perfectly match the PAM sequence NGG and the 8-12 base “seed sequence” at the 3′ end of the gRNA. According to certain aspects, perfect match is not required of the remaining 8-12 bases. According to certain aspects, Cas9 will function with single mismatches at the 5′ end. According to certain aspects, the target locus's underlying chromatin structure and epigenetic state may affect efficiency of Cas9 function. According to certain aspects, Cas9 homologs having higher specificity are included as useful enzymes. One of skill in the art will be able to identify or engineer suitable Cas9 homologs. According to one aspect, CRISPR-targetable sequences include those having different PAM requirements (see reference (9)), or directed evolution. According to one aspect, inactivating one of the Cas9 nuclease domains increases the ratio of HR to NHEJ and may reduce toxicity (FIGS. 3A-1 and 3A-2, FIG. 5) (4, 5), while inactivating both domains may enable Cas9 to function as a retargetable DNA binding protein. Embodiments of the present disclosure have broad utility in synthetic biology (see references (21, 22)), the direct and multiplexed perturbation of gene networks (see references (13, 23)), and targeted ex vivo (see references (24-26)) and in vivo gene therapy (see reference (27)).

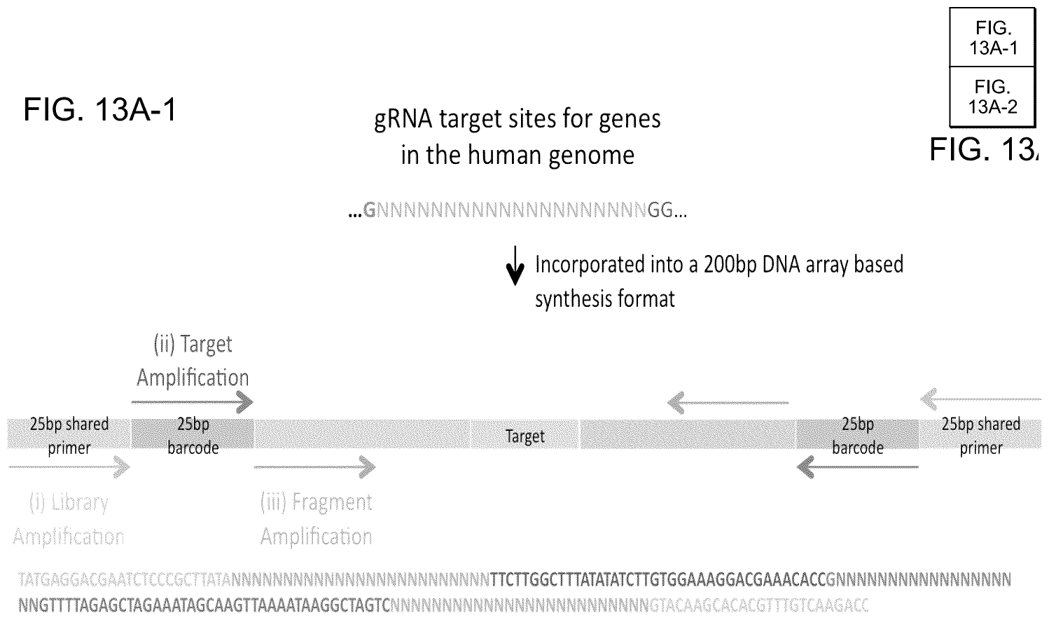

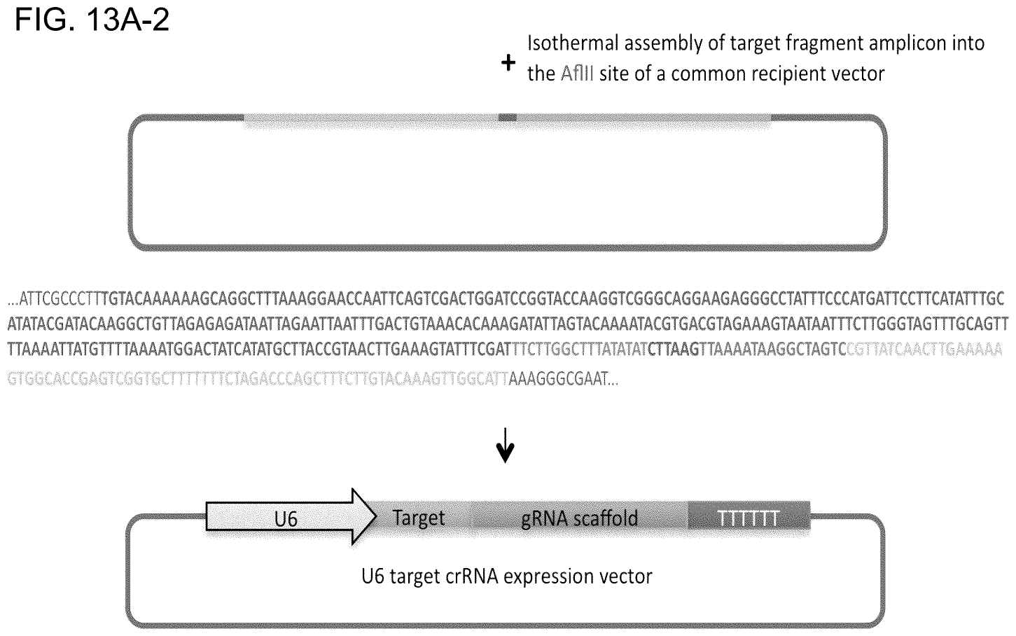

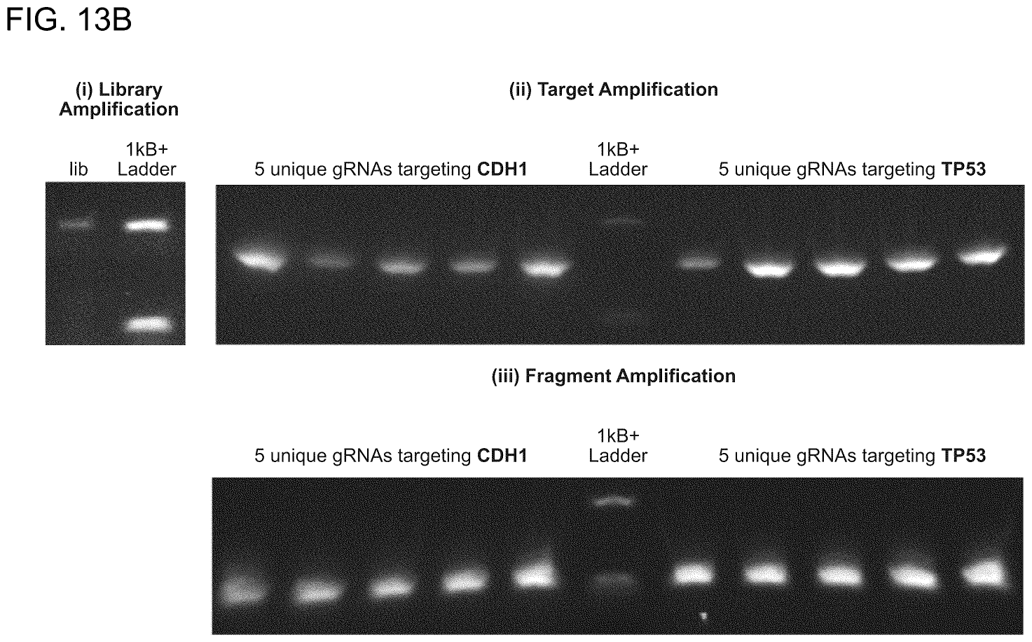

According to certain aspects, a “re-engineerable organism” is provided as a model system for biological discovery and in vivo screening. According to one aspect, a “re-engineerable mouse” bearing an inducible Cas9 transgene is provided, and localized delivery (using adeno-associated viruses, for example) of libraries of gRNAs targeting multiple genes or regulatory elements allow one to screen for mutations that result in the onset of tumors in the target tissue type. Use of Cas9 homologs or nuclease-null variants bearing effector domains (such as activators) allow one to multiplex activate or repress genes in vivo. According to this aspect, one could screen for factors that enable phenotypes such as: tissue-regeneration, trans-differentiation etc. According to certain aspects, (a) use of DNA-arrays enables multiplex synthesis of defined gRNA libraries (refer FIGS. 13A and 13B); and (b) gRNAs being small in size (refer FIG. 3b) are packaged and delivered using a multitude of non-viral or viral delivery methods.

According to one aspect, the lower toxicities observed with “nickases” for genome engineering applications is achieved by inactivating one of the Cas9 nuclease domains, either the nicking of the DNA strand base-paired with the RNA or nicking its complement. Inactivating both domains allows Cas9 to function as a retargetable DNA binding protein. According to one aspect, the Cas9 retargetable DNA binding protein is attached
(a) to transcriptional activation or repression domains for modulating target gene expression, including but not limited to chromatin remodeling, histone modification, silencing, insulation, direct interactions with the transcriptional machinery;
(b) to nuclease domains such as FokI to enable ‘highly specific’ genome editing contingent upon dimerization of adjacent gRNA-Cas9 complexes;
(c) to fluorescent proteins for visualizing genomic loci and chromosome dynamics; or
(d) to other fluorescent molecules such as protein or nucleic acid bound organic fluorophores, quantum dots, molecular beacons and echo probes or molecular beacon replacements;
(e) to multivalent ligand-binding protein domains that enable programmable manipulation of genome-wide 3D architecture.

According to one aspect, the transcriptional activation and repression components can employ CRISPR systems naturally or synthetically orthogonal, such that the gRNAs only bind to the activator or repressor class of Cas. This allows a large set of gRNAs to tune multiple targets.

According to certain aspects, the use of gRNAs provide the ability to multiplex than mRNAs in part due to the smaller size—100 vs. 2000 nucleotide lengths respectively. This is particularly valuable when nucleic acid delivery is size limited, as in viral packaging. This enables multiple instances of cleavage, nicking, activation, or repression—or combinations thereof. The ability to easily target multiple regulatory targets allows the coarse-or-fine-tuning or regulatory networks without being constrained to the natural regulatory circuits downstream of specific regulatory factors (e.g. the 4 mRNAs used in reprogramming fibroblasts into IPSCs). Examples of multiplexing applications include:
1. Establishing (major and minor) histocompatibility alleles, haplotypes, and genotypes for human (or animal) tissue/organ transplantation. This aspect results e.g. in HLA homozygous cell lines or humanized animal breeds—or—a set of gRNAs capable of superimposing such HLA alleles onto an otherwise desirable cell lines or breeds.
2. Multiplex cis-regulatory element (CRE=signals for transcription, splicing, translation, RNA and protein folding, degradation, etc.) mutations in a single cell (or a collection of cells) can be used for efficiently studying the complex sets of regulatory interaction that can occur in normal development or pathological, synthetic or pharmaceutical scenarios. According to one aspect, the CREs are (or can be made) somewhat orthogonal (i.e. low cross talk) so that many can be tested in one setting—e.g. in an expensive animal embryo time series. One exemplary application is with RNA fluorescent in situ sequencing (FISSeq).
3. Multiplex combinations of CRE mutations and/or epigenetic activation or repression of CREs can be used to alter or reprogram iPSCs or ESCs or other stem cells or non-stem cells to any cell type or combination of cell types for use in organs-on-chips or other cell and organ cultures for purposes of testing pharmaceuticals (small molecules, proteins, RNAs, cells, animal, plant or microbial cells, aerosols and other delivery methods), transplantation strategies, personalization strategies, etc.
4. Making multiplex mutant human cells for use in diagnostic testing (and/or DNA sequencing) for medical genetics. To the extent that the chromosomal location and context of a human genome allele (or epigenetic mark) can influence the accuracy of a clinical genetic diagnosis, it is important to have alleles present in the correct location in a reference genome—rather than in an ectopic (aka transgenic) location or in a separate piece of synthetic DNA. One embodiment is a series of independent cell lines one per each diagnostic human SNP, or structural variant. Alternatively, one embodiment includes multiplex sets of alleles in the same cell. In some cases multiplex changes in one gene (or multiple genes) will be desirable under the assumption of independent testing. In other cases, particular haplotype combinations of alleles allows testing of sequencing (genotyping) methods which accurately establish haplotype phase (i.e. whether one or both copies of a gene are affected in an individual person or somatic cell type.
5. Repetitive elements or endogenous viral elements can be targeted with engineered Cas+gRNA systems in microbes, plants, animals, or human cells to reduce deleterious transposition or to aid in sequencing or other analytic genomic/transcriptomic/proteomic/diagnostic tools (in which nearly identical copies can be problematic).

The following references identified by number in the foregoing section are hereby incorporated by reference in their entireties.
1. B. Wiedenheft, S. H. Sternberg, J. A. Doudna, Nature 482, 331 (Feb. 16, 2012).
2. D. Bhaya, M. Davison, R. Barrangou, Annual review of genetics 45, 273 (2011).
3. M. P. Terns, R. M. Terns, Current opinion in microbiology 14, 321 (June, 2011).
4. M. Jinek et al., Science 337, 816 (Aug. 17, 2012).
5. G. Gasiunas, R. Barrangou, P. Horvath, V. Siksnys, Proceedings of the National Academy of Sciences of the United States of America 109, E2579 (Sep. 25, 2012).
6. R. Sapranauskas et al., Nucleic acids research 39, 9275 (November, 2011).
7. T. R. Brummelkamp, R. Bernards, R. Agami, Science 296, 550 (Apr. 19, 2002).
8. M. Miyagishi, K. Taira, Nature biotechnology 20, 497 (May, 2002).
9. E. Deltcheva et al., Nature 471, 602 (Mar. 31, 2011).
10. J. Zou, P. Mali, X. Huang, S. N. Dowey, L. Cheng, Blood 118, 4599 (Oct. 27, 2011).
11. N. E. Sanjana et al., Nature protocols 7, 171 (January, 2012).
12. J. H. Lee et al., PLOS Genet 5, e1000718 (November, 2009).
13. D. Hockemeyer et al., Nature biotechnology 27, 851 (September, 2009).
14. S. Kosuri et al., Nature biotechnology 28, 1295 (December, 2010).
15. V. Pattanayak, C. L. Ramirez, J. K. Joung, D. R. Liu, Nature methods 8, 765 (September, 2011).
16. N. M. King, O. Cohen-Haguenauer, Molecular therapy: the journal of the American Society of Gene Therapy 16, 432 (March, 2008).
17. Y. G. Kim, J. Cha, S. Chandrasegaran, Proceedings of the National Academy of Sciences of the United States of America 93, 1156 (Feb. 6, 1996).
18. E. J. Rebar, C. O. Pabo, Science 263, 671 (Feb. 4, 1994).
19. J. Boch et al., Science 326, 1509 (Dec. 11, 2009).
20. M. J. Moscou, A. J. Bogdanove, Science 326, 1501 (Dec. 11, 2009).
21. A. S. Khalil, J. J. Collins, Nature reviews. Genetics 11, 367 (May, 2010).
22. P. E. Purnick, R. Weiss, Nature reviews. Molecular cell biology 10, 410 (June, 2009).
23. J. Zou et al., Cell stem cell 5, 97 (Jul. 2, 2009).
24. N. Holt et al., Nature biotechnology 28, 839 (August, 2010).
25. F. D. Urnov et al., Nature 435, 646 (Jun. 2, 2005).
26. A. Lombardo et al., Nature biotechnology 25, 1298 (November, 2007).
27. H. Li et al., Nature 475, 217 (Jul. 14, 2011).

The following examples are set forth as being representative of the present disclosure. These examples are not to be construed as limiting the scope of the present disclosure as these and other equivalent embodiments will be apparent in view of the present disclosure, figures and accompanying claims.

### Example I

### The Type II CRISPR-Cas System

According to one aspect, embodiments of the present disclosure utilize short RNA to identify foreign nucleic acids for activity by a nuclease in a eukaryotic cell. According to a certain aspect of the present disclosure, a eukaryotic cell is altered to include within its genome nucleic acids encoding one or more short RNA and one or more nucleases which are activated by the binding of a short RNA to a target DNA sequence. According to certain aspects, exemplary short RNA/enzyme systems may be identified within bacteria or archaea, such as (CRISPR)/CRISPR-associated (Cas) systems that use short RNA to direct degradation of foreign nucleic acids. CRISPR (“clustered regularly interspaced short palindromic repeats”) defense involves acquisition and integration of new targeting “spacers” from invading virus or plasmid DNA into the CRISPR locus, expression and processing of short guiding CRISPR RNAs (crRNAs) consisting of spacer-repeat units, and cleavage of nucleic acids (most commonly DNA) complementary to the spacer.

Three classes of CRISPR systems are generally known and are referred to as Type I, Type II or Type III). According to one aspect, a particular useful enzyme according to the present disclosure to cleave dsDNA is the single effector enzyme, Cas9, common to Type II. (See reference (1)). Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. According to one aspect, eukaryotic cells of the present disclosure are engineered to avoid use of RNase III and the crRNA processing in general. See reference (2).

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see reference (3)) and NNAGAAW (see reference (4)), respectively, while different S. mutans systems tolerate NGG or NAAR (see reference (5)). Bioinformatic analyses have generated extensive databases of CRISPR loci in a variety of bacteria that may serve to identify additional useful PAMs and expand the set of CRISPR-targetable sequences (see references (6, 7)). In S. thermophilus, Cas9 generates a blunt-ended double-stranded break 3 bp prior to the 3′ end of the protospacer (see reference (8)), a process mediated by two catalytic domains in the Cas9 protein: an HNH domain that cleaves the complementary strand of the DNA and a RuvC-like domain that cleaves the non-complementary strand (See FIG. 1A and FIGS. 3A-1 and 3A-2). While the S. pyogenes system has not been characterized to the same level of precision, DSB formation also occurs towards the 3′ end of the protospacer. If one of the two nuclease domains is inactivated, Cas9 will function as a nickase in vitro (see reference (2)) and in human cells (see FIG. 5).

According to one aspect, the specificity of gRNA-directed Cas9 cleavage is used as a mechanism for genome engineering in a eukaryotic cell. According to one aspect, hybridization of the gRNA need not be 100 percent in order for the enzyme to recognize the gRNA/DNA hybrid and affect cleavage. Some off-target activity could occur. For example, the S. pyogenes system tolerates mismatches in the first 6 bases out of the 20 bp mature spacer sequence in vitro. According to one aspect, greater stringency may be beneficial in vivo when potential off-target sites matching (last 14 bp) NGG exist within the human reference genome for the gRNAs. The effect of mismatches and enzyme activity in general are described in references (9), (2), (10), and (4).

According to certain aspects, specificity may be improved. When interference is sensitive to the melting temperature of the gRNA-DNA hybrid, AT-rich target sequences may have fewer off-target sites. Carefully choosing target sites to avoid pseudo-sites with at least 14 bp matching sequences elsewhere in the genome may improve specificity. The use of a Cas9 variant requiring a longer PAM sequence may reduce the frequency of off-target sites. Directed evolution may improve Cas9 specificity to a level sufficient to completely preclude off-target activity, ideally requiring a perfect 20 bp gRNA match with a minimal PAM. Accordingly, modification to the Cas9 protein is a representative embodiment of the present disclosure. As such, novel methods permitting many rounds of evolution in a short timeframe (see reference (11) and envisioned. CRISPR systems useful in the present disclosure are described in references (12, 13).

### Example II

### Plasmid Construction

The Cas9 gene sequence was human codon optimized and assembled by hierarchical fusion PCR assembly of 9 500 bp gBlocks ordered from IDT. FIGS. 3A-1 and 3A-2 for the engineered type II CRISPR system for human cells shows the expression format and full sequence of the cas9 gene insert. The RuvC-like and HNH motifs, and the C-terminus SV40 NLS are respectively highlighted by blue, brown and orange colors. Cas9_D10A was similarly constructed. The resulting full-length products were cloned into the pcDNA3.3-TOPO vector (Invitrogen). The target gRNA expression constructs were directly ordered as individual 455 bp gBlocks from IDT and either cloned into the pCR-BluntII-TOPO vector (Invitrogen) or pcr amplified. FIG. 3B shows the U6 promoter based expression scheme for the guide RNAs and predicted RNA transcript secondary structure. The use of the U6 promoter constrains the 1^{st} position in the RNA transcript to be a ‘G’ and thus all genomic sites of the form GN20GG can be targeted using this approach. FIG. 3C shows the 7 gRNAs used.

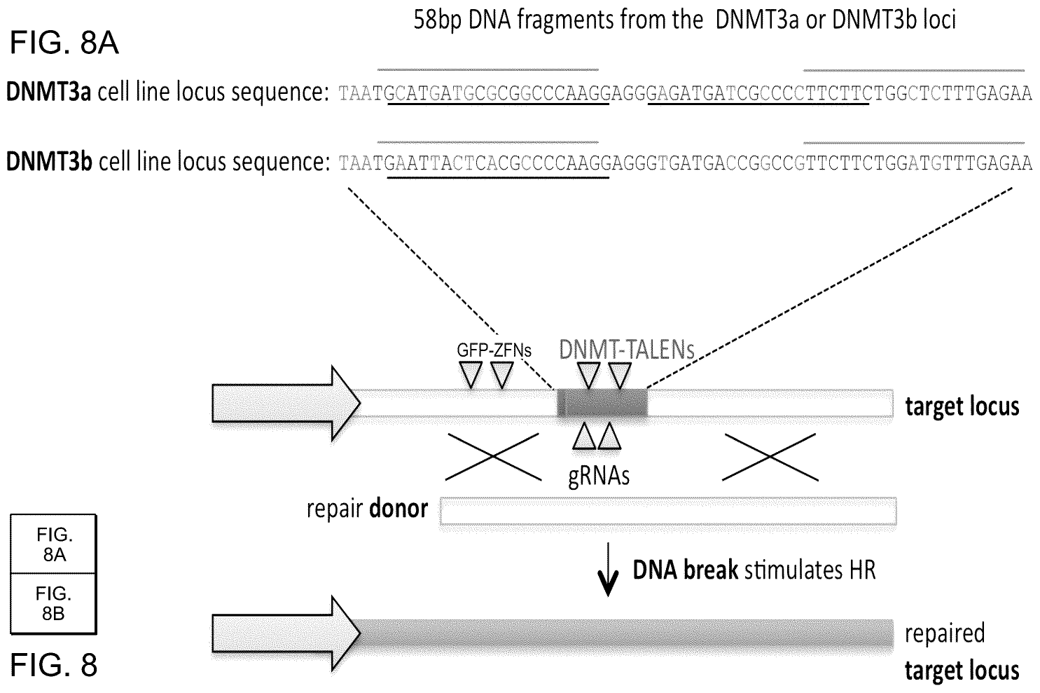

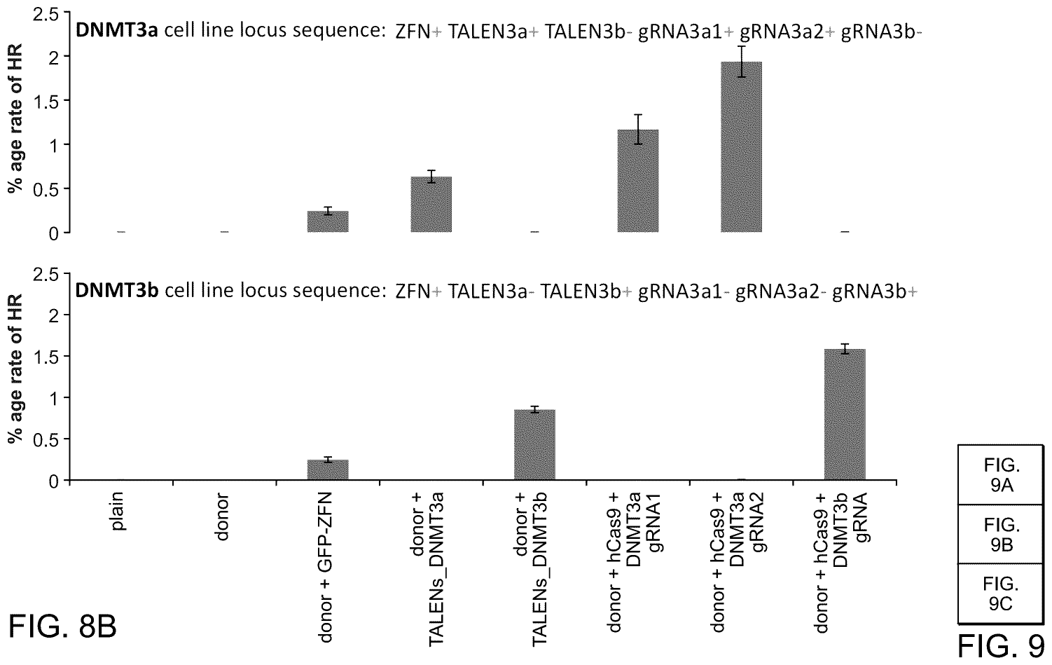

The vectors for the HR reporter assay involving a broken GFP were constructed by fusion PCR assembly of the GFP sequence bearing the stop codon and 68 bp AAVS1 fragment (or mutants thereof; see FIGS. 6A and 6B), or 58 bp fragments from the DNMT3a and DNMT3b genomic loci (see FIG. 8A) assembled into the EGIP lentivector from Addgene (plasmid #26777). These lentivectors were then used to establish the GFP reporter stable lines. TALENs used in this study were constructed using the protocols described in (14). All DNA reagents developed in this study are available at Addgene.

### Example III

### Cell Culture

PGP1 iPS cells were maintained on Matrigel (BD Biosciences)-coated plates in mTeSR1 (Stemcell Technologies). Cultures were passaged every 5-7 d with TrypLE Express (Invitrogen). K562 cells were grown and maintained in RPMI (Invitrogen) containing 15% FBS. HEK 293T cells were cultured in Dulbecco's modified Eagle's medium (DMEM, Invitrogen) high glucose supplemented with 10% fetal bovine serum (FBS, Invitrogen), penicillin/streptomycin (pen/strep, Invitrogen), and non-essential amino acids (NEAA, Invitrogen). All cells were maintained at 37° C. and 5% CO₂ in a humidified incubator.

### Example IV

Gene Targeting of PGP1 iPS, K562 and 293 Ts

PGP1 iPS cells were cultured in Rho kinase (ROCK) inhibitor (Calbiochem) 2 h before nucleofection. Cells were harvest using TrypLE Express (Invitrogen) and 2×10⁶ cells were resuspended in P3 reagent (Lonza) with 1 μg Cas9 plasmid, 1 μg gRNA and/or 1 μg DNA donor plasmid, and nucleofected according to manufacturer's instruction (Lonza). Cells were subsequently plated on an mTeSR1-coated plate in mTeSR1 medium supplemented with ROCK inhibitor for the first 24 h. For K562s, 2×10⁶ cells were resuspended in SF reagent (Lonza) with 1 μg Cas9 plasmid, 1 μg gRNA and/or 1 μg DNA donor plasmid, and nucleofected according to manufacturer's instruction (Lonza). For 293 Ts, 0.1×10⁶ cells were transfected with 1 μg Cas9 plasmid, 1 μg gRNA and/or 1 μg DNA donor plasmid using Lipofectamine 2000 as per the manufacturer's protocols. The DNA donors used for endogenous AAVS1 targeting were either a dsDNA donor (FIG. 2C) or a 90 mer oligonucleotide. The former has flanking short homology arms and a SA-2A-puromycin-CaGGS-eGFP cassette to enrich for successfully targeted cells.

The targeting efficiency was assessed as follows. Cells were harvested 3 days after nucleofection and the genomic DNA of ˜1×10⁶ cells was extracted using prepGEM (ZyGEM). PCR was conducted to amplify the targeting region with genomic DNA derived from the cells and amplicons were deep sequenced by MiSeq Personal Sequencer (Illumina) with coverage >200,000 reads. The sequencing data was analyzed to estimate NHEJ efficiencies. The reference AAVS1 sequence analyzed is:

(SEQ ID NO: 1)
CACTTCAGGACAGCATGTTTGCTGCCTCCAGGGATCCTGTGTCCCCGAGC
TGGGACCACCTTATATTCCCAGGGCCGGTTAATGTGGCTCTGGTTCTGGG
TACTTTTATCTGTCCCCTCCACCCCACAGTGGGGCCACTAGGGACAGGAT
TGGTGACAGAAAAGCCCCATCCTTAGGCCTCCTCCTTCCTAGTCTCCTGA
TATTGGGTCTAACCCCCACCTCCTGTTAGGCAGATTCCTTATCTGGTGAC
ACACCCCCATTTCCTGGA

The PCR primers for amplifying the targeting regions in the human genome are:

AAVS1-R
(SEQ ID NO: 2)
CTCGGCATTCCTGCTGAACCGCTCTTCCGATCTacaggaggtgggggtta
gac
AAVS1-F.1
(SEQ ID NO: 3)
ACACTCTTTCCCTACACGACGCTCTTCCGATCTCGTGATtatattcccag
ggccggtta
AAVS1-F.2
(SEQ ID NO: 4)
ACACTCTTTCCCTACACGACGCTCTTCCGATCTACATCGtatattcccag
ggccggtta
AAVS1-F.3
(SEQ ID NO: 5)
ACACTCTTTCCCTACACGACGCTCTTCCGATCTGCCTAAtatattcccag
ggccggtta
AAVS1-F.4
(SEQ ID NO: 6)
ACACTCTTTCCCTACACGACGCTCTTCCGATCTTGGTCAtatattcccag
ggccggtta
AAVS1-F.5
(SEQ ID NO: 7)
ACACTCTTTCCCTACACGACGCTCTTCCGATCTCACTGTtatattcccag
ggccggtta
AAVS1-F.6
(SEQ ID NO: 8)
ACACTCTTTCCCTACACGACGCTCTTCCGATCTATTGGCtatattcccag
ggccggtta
AAVS1-F.7
(SEQ ID NO: 9)
ACACTCTTTCCCTACACGACGCTCTTCCGATCTGATCTGtatattcccag
ggccggtta
AAVS1-F.8
(SEQ ID NO: 10)
ACACTCTTTCCCTACACGACGCTCTTCCGATCTTCAAGTtatattcccag
ggccggtta
AAVS1-F.9
(SEQ ID NO: 11)
ACACTCTTTCCCTACACGACGCTCTTCCGATCTCTGATCtatattcccag
ggccggtta
AAVS1-F.10
(SEQ ID NO: 12)
ACACTCTTTCCCTACACGACGCTCTTCCGATCTAAGCTAtatattcccag
ggccggtta
AAVS1-F.11
(SEQ ID NO: 13)
ACACTCTTTCCCTACACGACGCTCTTCCGATCTGTAGCCtatattcccag
ggccggtta
AAVS1-F.12
(SEQ ID NO: 14)
ACACTCTTTCCCTACACGACGCTCTTCCGATCTTACAAGtatattcccag
ggccggtta

To analyze the HR events using the DNA donor in FIG. 2C, the primers used were:

HR_AAVS1-F
(SEQ ID NO: 15)
CTGCCGTCTCTCTCCTGAGT
HR_Puro-R
(SEQ ID NO: 16)
GTGGGCTTGTACTCGGTCAT

### Example V

Bioinformatics Approach for Computing Human Exon CRISPR Targets and Methodology for their Multiplexed Synthesis

A set of gRNA gene sequences that maximally target specific locations in human exons but minimally target other locations in the genome were determined as follows. According to one aspect, maximally efficient targeting by a gRNA is achieved by 23 nt sequences, the 5′-most 20 nt of which exactly complement a desired location, while the three 3′-most bases must be of the form NGG. Additionally, the 5′-most nt must be a G to establish a pol-III transcription start site. However, according to (2), mispairing of the six 5′-most nt of a 20 bp gRNA against its genomic target does not abrogate Cas9-mediated cleavage so long as the last 14 nt pairs properly, but mispairing of the eight 5′-most nt along with pairing of the last 12 nt does, while the case of the seven 5-most nt mispairs and 13 3′ pairs was not tested. To be conservative regarding off-target effects, one condition was that the case of the seven 5′-most mispairs is, like the case of six, permissive of cleavage, so that pairing of the 3′-most 13 nt is sufficient for cleavage. To identify CRISPR target sites within human exons that should be cleavable without off-target cuts, all 23 bp sequences of the form 5′-GBBBB BBBBB BBBBB BBBBB NGG-3′ (form 1) were examined, where the B's represent the bases at the exon location, for which no sequence of the form 5′-NNNNN NNBBB BBBBB BBBBB NGG-3′ (form 2) existed at any other location in the human genome. Specifically, (i) a BED file of locations of coding regions of all RefSeq genes the GRCh37/hg19 human genome from the UCSC Genome Browser (15-17) was downloaded. Coding exon locations in this BED file comprised a set of 346089 mappings of RefSeq mRNA accessions to the hg19 genome. However, some RefSeq mRNA accessions mapped to multiple genomic locations (probable gene duplications), and many accessions mapped to subsets of the same set of exon locations (multiple isoforms of the same genes). To distinguish apparently duplicated gene instances and consolidate multiple references to the same genomic exon instance by multiple RefSeq isoform accessions, (ii) unique numerical suffixes to 705 RefSeq accession numbers that had multiple genomic locations were added, and (iii) the mergeBed function of BEDTools (18) (v2.16.2-zip-87e3926) was used to consolidate overlapping exon locations into merged exon regions. These steps reduced the initial set of 346089 RefSeq exon locations to 192783 distinct genomic regions. The hg19 sequence for all merged exon regions were downloaded using the UCSC Table Browser, adding 20 bp of padding on each end. (iv) Using custom perl code, 1657793 instances of form 1 were identified within this exonic sequence. (v) These sequences were then filtered for the existence of off-target occurrences of form 2: For each merged exon form 1 target, the 3′-most 13 bp specific (B) “core” sequences were extracted and, for each core generated the four 16 bp sequences 5′-BBB BBBBB BBBBB NGG-3′ (N=A, C, G, and T), and searched the entire hg19 genome for exact matches to these 6631172 sequences using Bowtie version 0.12.8 (19) using the parameters-1 16-v 0-k 2. Any exon target site for which there was more than a single match was rejected. Note that because any specific 13 bp core sequence followed by the sequence NGG confers only 15 bp of specificity, there should be on average ˜5.6 matches to an extended core sequence in a random ˜3 Gb sequence (both strands). Therefore, most of the 1657793 initially identified targets were rejected; however 189864 sequences passed this filter. These comprise the set of CRISPR-targetable exonic locations in the human genome. The 189864 sequences target locations in 78028 merged exonic regions (˜40.5% of the total of 192783 merged human exon regions) at a multiplicity of ˜2.4 sites per targeted exonic region. To assess targeting at a gene level, RefSeq mRNA mappings were clustered so that any two RefSeq accessions (including the gene duplicates distinguished in (ii)) that overlap a merged exon region are counted as a single gene cluster, the 189864 exonic specific CRISPR sites target 17104 out of 18872 gene clusters (˜90.6% of all gene clusters) at a multiplicity of ˜11.1 per targeted gene cluster. (Note that while these gene clusters collapse RefSeq mRNA accessions that represent multiple isoforms of a single transcribed gene into a single entity, they will also collapse overlapping distinct genes as well as genes with antisense transcripts.) At the level of original RefSeq accessions, the 189864 sequences targeted exonic regions in 30563 out of a total of 43726 (˜69.9%) mapped RefSeq accessions (including distinguished gene duplicates) at a multiplicity of ˜6.2 sites per targeted mapped RefSeq accession.

According to one aspect, the database can be refined by correlating performance with factors, such as base composition and secondary structure of both gRNAs and genomic targets (20, 21), and the epigenetic state of these targets in human cell lines for which this information is available (22).

### Example VI

### Multiplex Synthesis

The target sequences were incorporated into a 200 bp format that is compatible for multiplex synthesis on DNA arrays (23, 24). According to one aspect the method allows for targeted retrieval of a specific or pools of gRNA sequences from the DNA array based oligonucleotide pool and its rapid cloning into a common expression vector (FIGS. 13A-1 and 13A-2). Specifically, a 12 k oligonucleotide pool from CustomArray Inc. was synthesized. Furthermore, gRNAs of choice from this library (FIG. 13B) were successfully retrieved. We observed an error rate of ˜4 mutations per 1000 bp of synthesized DNA.

### Example VII

RNA-guided genome editing requires both Cas9 and guide RNA for successful targeting Using the GFP reporter assay described in FIG. 1B, all possible combinations of the repair DNA donor, Cas9 protein, and gRNA were tested for their ability to effect successful HR (in 293 Ts). As shown in FIG. 4, GFP+ cells were observed only when all the 3 components were present, validating that these CRISPR components are essential for RNA-guided genome editing. Data is mean+/−SEM (N=3).

### Example VIII

Analysis of gRNA and Cas9 Mediated Genome Editing

The CRISPR mediated genome editing process was examined using either (A) a GFP reporter assay as described earlier results of which are shown in FIG. 5A, and (B) deep sequencing of the targeted loci (in 293 Ts), results of which are shown in FIG. 5B. As comparison, a D10A mutant for Cas9 was tested that has been shown in earlier reports to function as a nickase in in vitro assays. As shown in FIG. 5, both Cas9 and Cas9D10A can effect successful HR at nearly similar rates. Deep sequencing however confirms that while Cas9 shows robust NHEJ at the targeted loci, the D10A mutant has significantly diminished NHEJ rates (as would be expected from its putative ability to only nick DNA). Also, consistent with the known biochemistry of the Cas9 protein, NHEJ data confirms that most base-pair deletions or insertions occurred near the 3′ end of the target sequence: the peak is ˜3-4 bases upstream of the PAM site, with a median deletion frequency of ˜9-10 bp. Data is mean+/−SEM (N=3).

### Example IX

### RNA-Guided Genome Editing is Target Sequence Specific

Similar to the GFP reporter assay described in FIG. 1B, 3 293T stable lines each bearing a distinct GFP reporter construct were developed. These are distinguished by the sequence of the AAVS1 fragment insert (as indicated in the FIGS. 6A and 6B). One line harbored the wild-type fragment while the two other lines were mutated at 6 bp (highlighted in red). Each of the lines was then targeted by one of the following 4 reagents: a GFP-ZFN pair that can target all cell types since its targeted sequence was in the flanking GFP fragments and hence present in along cell lines; a AAVS1 TALEN that could potentially target only the wt-AAVS1 fragment since the mutations in the other two lines should render the left TALEN unable to bind their sites; the T1 gRNA which can also potentially target only the wt-AAVS1 fragment, since its target site is also disrupted in the two mutant lines; and finally the T2 gRNA which should be able to target all 3 cell lines since, unlike the T1 gRNA, its target site is unaltered among the 3 lines. ZFN modified all 3 cell types, the AAVS1 TALENs and the T1 gRNA only targeted the wt-AAVS1 cell type, and the T2 gRNA successfully targets all 3 cell types. These results together confirm that the guide RNA mediated editing is target sequence specific. Data is mean+/−SEM (N=3).

### Example X

### Guide RNAs Targeted to the GFP Sequence Enable Robust Genome Editing

In addition to the 2 gRNAs targeting the AAVS1 insert, two additional gRNAs targeting the flanking GFP sequences of the reporter described in FIG. 1B (in 293 Ts) were tested. As shown in FIG. 7, these gRNAs were also able to effect robust HR at this engineered locus. Data is mean+/−SEM (N=3).

### Example XI

### RNA-Guided Genome Editing is Target Sequence Specific, and Demonstrates Similar Targeting Efficiencies as ZFNs or TALENs

Similar to the GFP reporter assay described in FIG. 1B, two 293T stable lines each bearing a distinct GFP reporter construct were developed. These are distinguished by the sequence of the fragment insert (as indicated in FIG. 8A). One line harbored a 58 bp fragment from the DNMT3a gene while the other line bore a homologous 58 bp fragment from the DNMT3b gene. The sequence differences are highlighted in red. Each of the lines was then targeted by one of the following 6 reagents: a GFP-ZFN pair that can target all cell types since its targeted sequence was in the flanking GFP fragments and hence present in along cell lines; a pair of TALENs that potentially target either DNMT3a or DNMT3b fragments; a pair of gRNAs that can potentially target only the DNMT3a fragment; and finally a gRNA that should potentially only target the DNMT3b fragment. As indicated in FIG. 8B, the ZFN modified all 3 cell types, and the TALENs and gRNAs only their respective targets. Furthermore the efficiencies of targeting were comparable across the 6 targeting reagents. These results together confirm that RNA-guided editing is target sequence specific and demonstrates similar targeting efficiencies as ZFNs or TALENs. Data is mean+/−SEM (N=3).

### Example XII

### RNA-Guided NHEJ in Human iPS Cells

Human iPS cells (PGP1) were nucleofected with constructs indicated in the left panel of FIGS. 9A, 9B, and 9C. 4 days after nucleofection, NHEJ rate was measured by assessing genomic deletion and insertion rate at double-strand breaks (DSBs) by deep sequencing. Panel 1: Deletion rate detected at targeting region. Red dash lines: boundary of T1 RNA targeting site; green dash lines: boundary of T2 RNA targeting site. The deletion incidence at each nucleotide position was plotted in black lines and the deletion rate as the percentage of reads carrying deletions was calculated. Panel 2: Insertion rate detected at targeting region. Red dash lines: boundary of T1 RNA targeting site; green dash lines: boundary of T2 RNA targeting site. The incidence of insertion at the genomic location where the first insertion junction was detected was plotted in black lines and the insertion rate as the percentage of reads carrying insertions was calculated. Panel 3: Deletion size distribution. The frequencies of different size deletions among the whole NHEJ population was plotted. Panel 4: insertion size distribution. The frequencies of different sizes insertions among the whole NHEJ population was plotted. iPS targeting by both gRNAs is efficient (2-4%), sequence specific (as shown by the shift in position of the NHEJ deletion distributions), and reaffirming the results of FIG. 4, the NGS-based analysis also shows that both the Cas9 protein and the gRNA are essential for NHEJ events at the target locus.

### Example XIII

### RNA-Guided NHEJ in K562 Cells

K562 cells were nucleated with constructs indicated in the left panel of FIGS. 10A and 10B. 4 days after nucleofection, NHEJ rate was measured by assessing genomic deletion and insertion rate at DSBs by deep sequencing. Panel 1: Deletion rate detected at targeting region. Red dash lines: boundary of T1 RNA targeting site; green dash lines: boundary of T2 RNA targeting site. The deletion incidence at each nucleotide position was plotted in black lines and the deletion rate as the percentage of reads carrying deletions was calculated. Panel 2: Insertion rate detected at targeting region. Red dash lines: boundary of T1 RNA targeting site; green dash lines: boundary of T2 RNA targeting site. The incidence of insertion at the genomic location where the first insertion junction was detected was plotted in black lines and the insertion rate as the percentage of reads carrying insertions was calculated. Panel 3: Deletion size distribution. The frequencies of different size deletions among the whole NHEJ population was plotted. Panel 4: insertion size distribution. The frequencies of different sizes insertions among the whole NHEJ population was plotted. K562 targeting by both gRNAs is efficient (13-38%) and sequence specific (as shown by the shift in position of the NHEJ deletion distributions). Importantly, as evidenced by the peaks in the histogram of observed frequencies of deletion sizes, simultaneous introduction of both T1 and T2 guide RNAs resulted in high efficiency deletion of the intervening 19 bp fragment, demonstrating that multiplexed editing of genomic loci is also feasible using this approach.

### Example XIV

### RNA-Guided NHEJ in 293T Cells

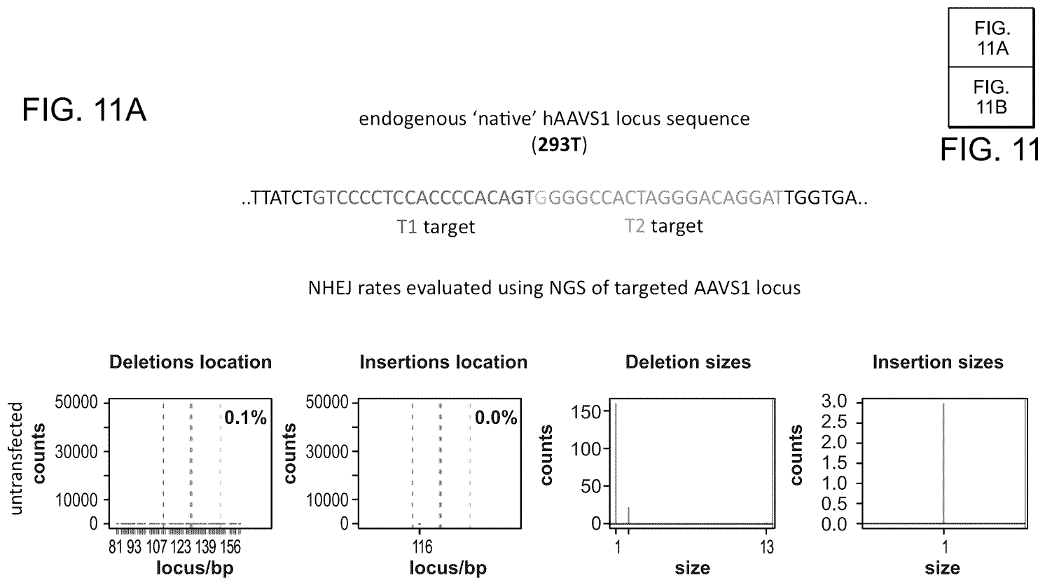

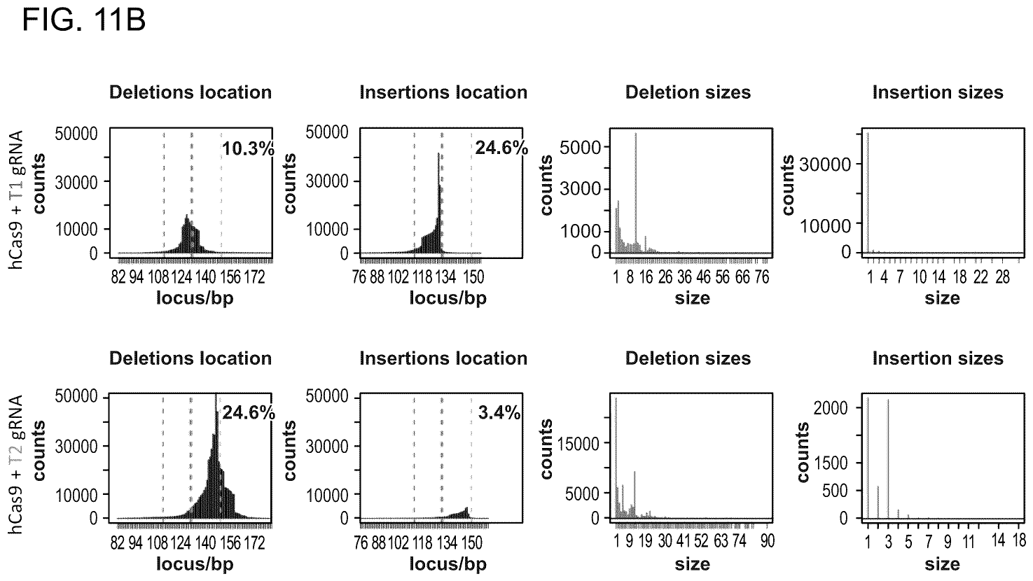

293T cells were transfected with constructs indicated in the left panel of FIGS. 11A and 11B. 4 days after nucleofection, NHEJ rate was measured by assessing genomic deletion and insertion rate at DSBs by deep sequencing. Panel 1: Deletion rate detected at targeting region. Red dash lines: boundary of T1 RNA targeting site; green dash lines: boundary of T2 RNA targeting site. The deletion incidence at each nucleotide position was plotted in black lines and the deletion rate as the percentage of reads carrying deletions was calculated. Panel 2: Insertion rate detected at targeting region. Red dash lines: boundary of T1 RNA targeting site; green dash lines: boundary of T2 RNA targeting site. The incidence of insertion at the genomic location where the first insertion junction was detected was plotted in black lines and the insertion rate as the percentage of reads carrying insertions was calculated. Panel 3: Deletion size distribution. The frequencies of different size deletions among the whole NHEJ population was plotted. Panel 4: insertion size distribution. The frequencies of different sizes insertions among the whole NHEJ population was plotted. 293T targeting by both gRNAs is efficient (10-24%) and sequence specific (as shown by the shift in position of the NHEJ deletion distributions).

### Example XV

HR at the Endogenous AAVS1 Locus Using Either a dsDNA Donor or a Short Oligonucleotide Donor

As shown in FIG. 12A, PCR screen (with reference to FIG. 2C) confirmed that 21/24 randomly picked 293T clones were successfully targeted. As shown in FIG. 12B, similar PCR screen confirmed 3/7 randomly picked PGP1-iPS clones were also successfully targeted. As shown in FIG. 12C, short 90 mer oligos could also effect robust targeting at the endogenous AAVS1 locus (shown here for K562 cells).

### Example XVI

### Methodology for Multiplex Synthesis, Retrieval and U6 Expression Vector Cloning of Guide RNAs Targeting Genes in the Human Genome

A resource of about 190 k bioinformatically computed unique gRNA sites targeting ˜40.5% of all exons of genes in the human genome was generated. As shown in FIG. 13A-1 and FIG. 13A-2, the gRNA target sites were incorporated into a 200 bp format that is compatible for multiplex synthesis on DNA arrays. Specifically, the design allows for (i) targeted retrieval of a specific or pools of gRNA targets from the DNA array oligonucleotide pool (through 3 sequential rounds of nested PCR as indicated in the figure schematic); and (ii) rapid cloning into a common expression vector which upon linearization using an AflII site serves as a recipient for Gibson assembly mediated incorporation of the gRNA insert fragment. As shown in FIG. 13B, the method was used to accomplish targeted retrieval of 10 unique gRNAs from a 12 k oligonucleotide pool synthesized by CustomArray Inc.

### Example XVII

### CRISPR Mediated RNA-Guided Transcriptional Activation

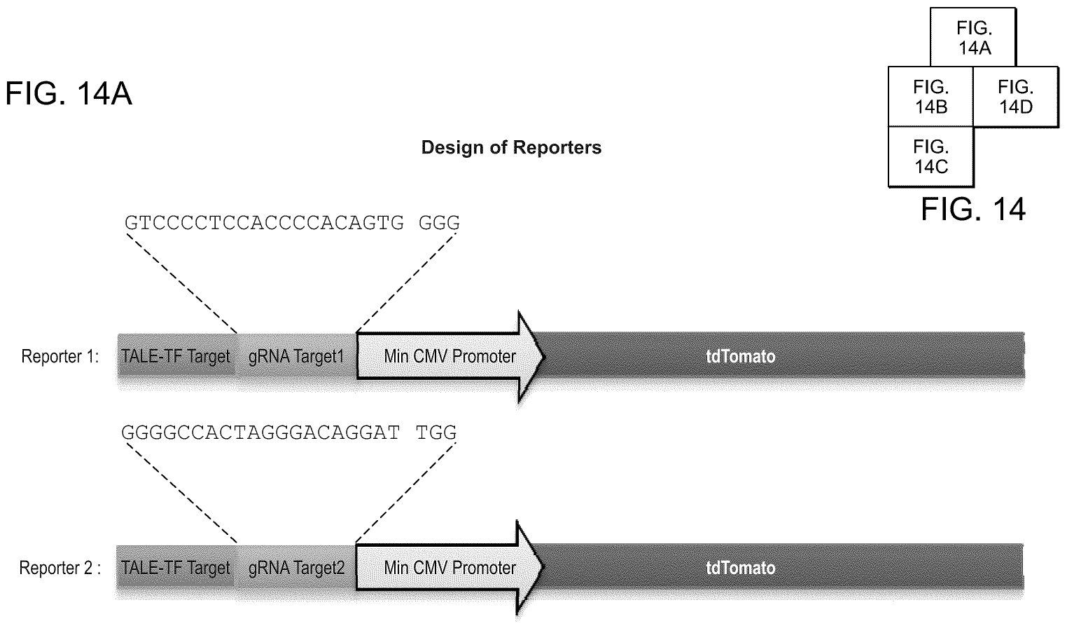

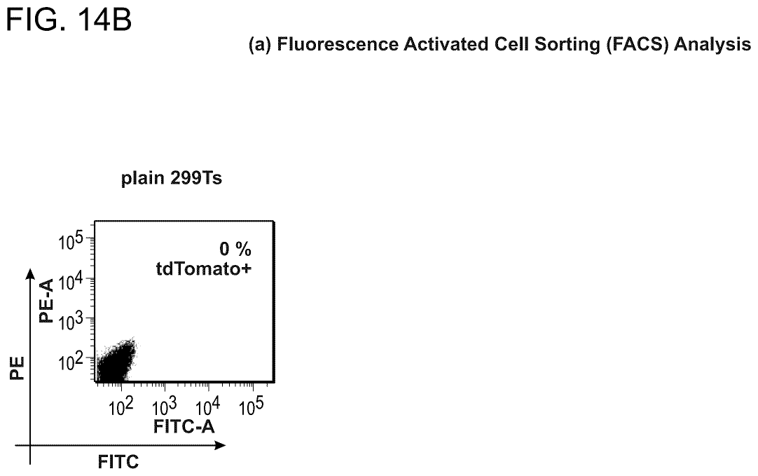

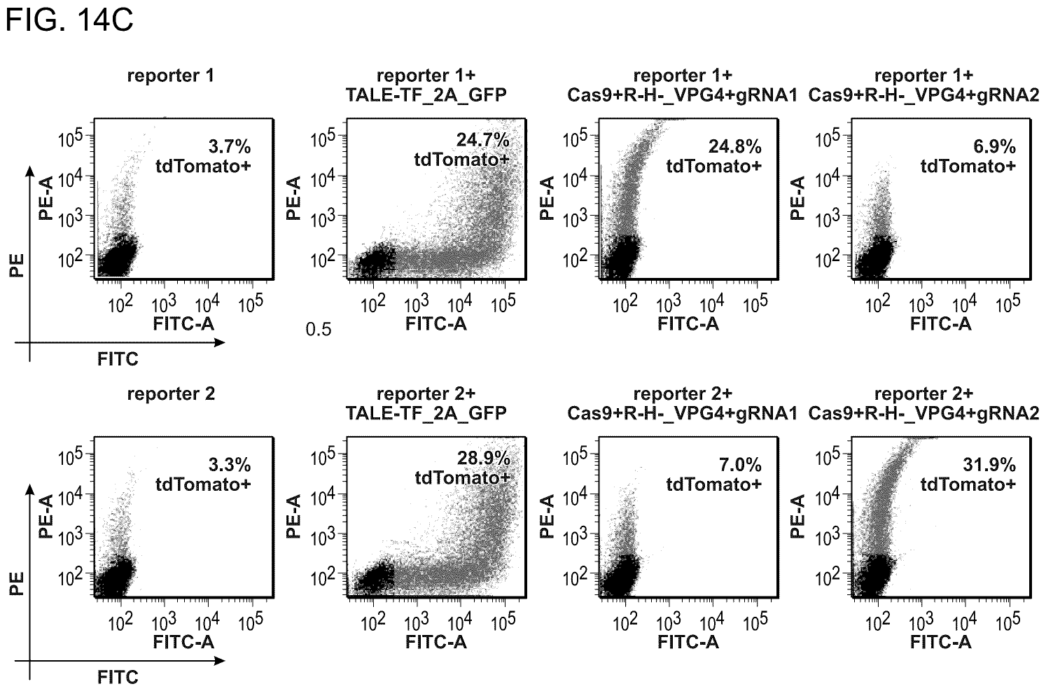

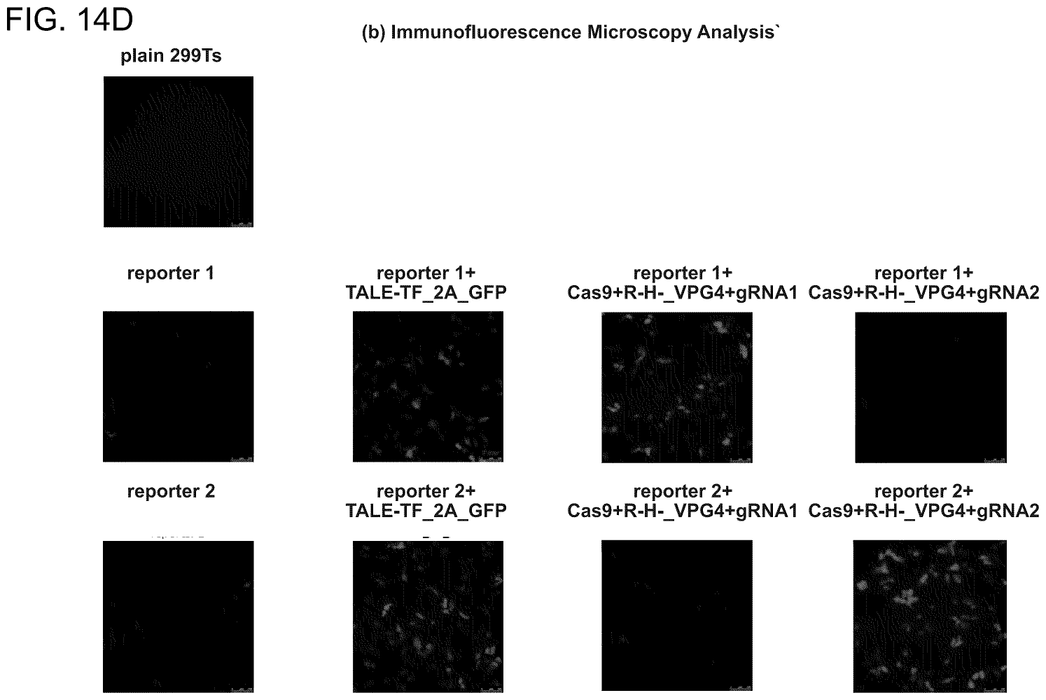

The CRISPR-Cas system has an adaptive immune defense system in bacteria and functions to ‘cleave’ invading nucleic acids. According to one aspect, the CRISPR-CAS system is engineered to function in human cells, and to ‘cleave’ genomic DNA. This is achieved by a short guide RNA directing a Cas9 protein (which has nuclease function) to a target sequence complementary to the spacer in the guide RNA. The ability to ‘cleave’ DNA enables a host of applications related to genome editing, and also targeted genome regulation. Towards this, the Cas9 protein was mutated to make it nuclease-null by introducing mutations that are predicted to abrogate coupling to Mg2+ (known to be important for the nuclease functions of the RuvC-like and HNH-like domains): specifically, combinations of D10A, D839A, H840A and N863A mutations were introduced. The thus generated Cas9 nuclease-null protein (as confirmed by its ability to not cut DNA by sequencing analysis) and hereafter referred to as Cas9R-H−, was then coupled to a transcriptional activation domain, here VP64, enabling the CRISPR-cas system to function as a RNA guided transcription factor (see FIGS. 14A-D). The Cas9R-H−+VP64 fusion enables RNA-guided transcriptional activation at the two reporters shown. Specifically, both FACS analysis and immunofluorescence imaging demonstrates that the protein enables gRNA sequence specific targeting of the corresponding reporters, and furthermore, the resulting transcription activation as assayed by expression of a dTomato fluorescent protein was at levels similar to those induced by a convention TALE-VP64 fusion protein.

### Example XVIII

gRNA Sequence Flexibility and Applications Thereof

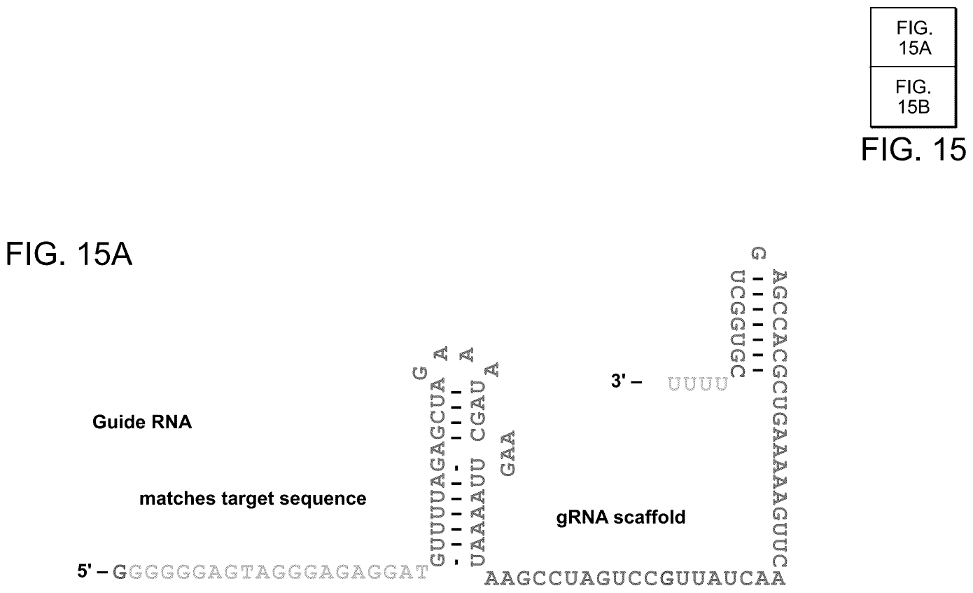

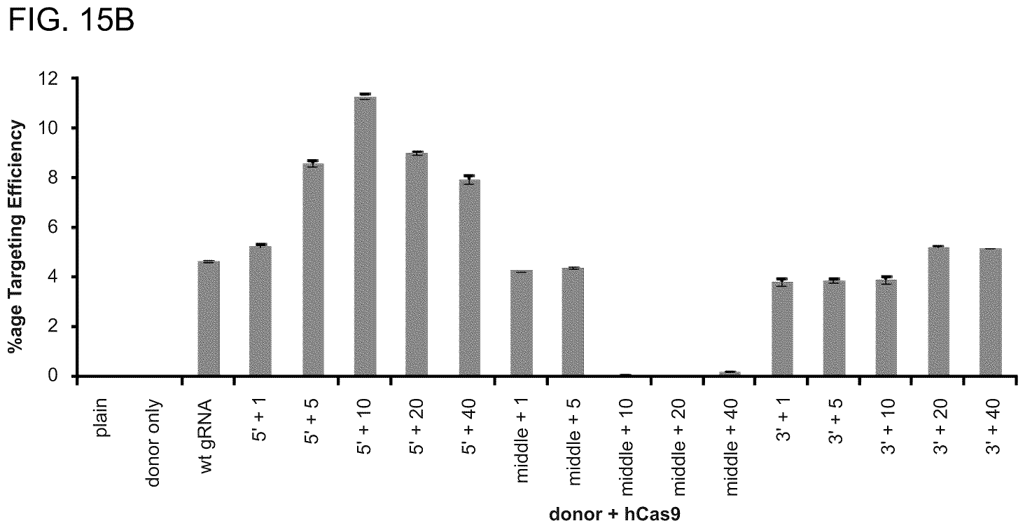

Flexibility of the gRNA scaffold sequence to designer sequence insertions was determined by systematically assaying for a range of the random sequence insertions on the 5′, middle and 3′ portions of the gRNA: specifically, 1 bp, 5 bp, 10 bp, 20 bp, and 40 bp inserts were made in the gRNA sequence at the 5′, middle, and 3′ ends of the gRNA (the exact positions of the insertion are highlighted in ‘red’ in FIG. 15A). This gRNA was then tested for functionality by its ability to induce HR in a GFP reporter assay (as described herein). It is evident that gRNAs are flexible to sequence insertions on the 5′ and 3′ ends (as measured by retained HR inducing activity). Accordingly, aspects of the present disclosure are directed to tagging of small-molecule responsive RNA aptamers that may trigger onset of gRNA activity, or gRNA visualization. Additionally, aspects of the present disclosure are directed to tethering of ssDNA donors to gRNAs via hybridization, thus enabling coupling of genomic target cutting and immediate physical localization of repair template which can promote homologous recombination rates over error-prone non-homologous end-joining.

The following references identified in the Examples section by number are hereby incorporated by reference in their entireties for all purposes.

### REFERENCES

ADDIN EN.REFLIST 1. K. S. Makarova et al., Evolution and classification of the CRISPR-Cas systems. Nature reviews. Microbiology 9, 467 (June, 2011).
2. M. Jinek et al., A programmable dual-RNA-guided DNA endonuclease in adaptive bacterial immunity. Science 337, 816 (Aug. 17, 2012).
3. P. Horvath, R. Barrangou, CRISPR/Cas, the immune system of bacteria and archaea. Science 327, 167 (Jan. 8, 2010).
4. H. Deveau et al., Phage response to CRISPR-encoded resistance in Streptococcus thermophilus. Journal of bacteriology 190, 1390 (February, 2008).
5. J. R. van der Ploeg, Analysis of CRISPR in Streptococcus mutans suggests frequent occurrence of acquired immunity against infection by M102-like bacteriophages. Microbiology 155, 1966 (June, 2009).
6. M. Rho, Y. W. Wu, H. Tang, T. G. Doak, Y. Ye, Diverse CRISPRs evolving in human microbiomes. PLOS genetics 8, e1002441 (2012).
7. D. T. Pride et al., Analysis of streptococcal CRISPRs from human saliva reveals substantial sequence diversity within and between subjects over time. Genome research 21, 126 (January, 2011).
8. G. Gasiunas, R. Barrangou, P. Horvath, V. Siksnys, Cas9-crRNA ribonucleoprotein complex mediates specific DNA cleavage for adaptive immunity in bacteria. Proceedings of the National Academy of Sciences of the United States of America 109, E2579 (Sep. 25, 2012).
9. R. Sapranauskas et al., The Streptococcus thermophilus CRISPR/Cas system provides immunity in Escherichia coli. Nucleic acids research 39, 9275 (November, 2011).
10. J. E. Garneau et al., The CRISPR/Cas bacterial immune system cleaves bacteriophage and plasmid DNA. Nature 468, 67 (Nov. 4, 2010).
11. K. M. Esvelt, J. C. Carlson, D. R. Liu, A system for the continuous directed evolution of biomolecules. Nature 472, 499 (Apr. 28, 2011).
12. R. Barrangou, P. Horvath, CRISPR: new horizons in phage resistance and strain identification. Annual review of food science and technology 3, 143 (2012).
13. B. Wiedenheft, S. H. Sternberg, J. A. Doudna, RNA-guided genetic silencing systems in bacteria and archaea. Nature 482, 331 (Feb. 16, 2012).
14. N. E. Sanjana et al., A transcription activator-like effector toolbox for genome engineering. Nature protocols 7, 171 (January, 2012).
15. W. J. Kent et al., The human genome browser at UCSC. Genome Res 12, 996 (June, 2002).
16. T. R. Dreszer et al., The UCSC Genome Browser database: extensions and updates 2011. Nucleic Acids Res 40, D918 (January, 2012).
17. D. Karolchik et al., The UCSC Table Browser data retrieval tool. Nucleic Acids Res 32, D493 (Jan. 1, 2004).
18. A. R. Quinlan, I. M. Hall, BEDTools: a flexible suite of utilities for comparing genomic features. Bioinformatics 26, 841 (Mar. 15, 2010).
19. B. Langmead, C. Trapnell, M. Pop, S. L. Salzberg, Ultrafast and memory-efficient alignment of short DNA sequences to the human genome. Genome Biol 10, R25 (2009).
20. R. Lorenz et al., ViennaRNA Package 2.0. Algorithms for molecular biology: AMB 6, 26 (2011).
21. D. H. Mathews, J. Sabina, M. Zuker, D. H. Turner, Expanded sequence dependence of thermodynamic parameters improves prediction of RNA secondary structure. Journal of molecular biology 288, 911 (May 21, 1999).
22. R. E. Thurman et al., The accessible chromatin landscape of the human genome. Nature 489, 75 (Sep. 6, 2012).
23. S. Kosuri et al., Scalable gene synthesis by selective amplification of DNA pools from high-fidelity microchips. Nature biotechnology 28, 1295 (December, 2010).
24. Q. Xu, M. R. Schlabach, G. J. Hannon, S. J. Elledge, Design of 240,000 orthogonal 25 mer DNA barcode probes. Proceedings of the National Academy of Sciences of the United States of America 106, 2289 (Feb. 17, 2009).

1.-14. (canceled)

15. A nucleic acid comprising a nucleotide sequence encoding a single molecule DNA-targeting RNA, wherein the single molecule DNA-targeting RNA comprises, in 5′ to 3′ order:
(i) a targeter-RNA comprising a first nucleotide sequence that is complementary to a target sequence of a target DNA, and a second nucleotide sequence that hybridizes with an activator-RNA, wherein the first and second nucleotide sequences are heterologous to one another; and
(ii) the activator-RNA, which comprises a duplex-forming segment that comprises a nucleotide sequence that hybridizes with the targeter-RNA to form a double-stranded duplex, wherein (i) and (ii) are covalently linked by intervening nucleotides, and wherein the single molecule DNA-targeting RNA is capable of forming a complex with a Cas9 protein and guiding the complex to the target sequence to cleave the target DNA,
wherein the nucleotide sequence encoding the single molecule DNA-targeting RNA is operably linked to a promoter heterologous to the second nucleotide sequence.

16. The nucleic acid of claim 15, wherein said nucleotide sequence of the duplex-forming segment of the activator-RNA comprises a sequence having 100% identity to the tracrRNA sequence UAGCAAGUUAAAAU.

17. The nucleic acid of claim 15, wherein the activator-RNA comprises the 26 nucleotide tracrRNA sequence UAGCAAGUUAAAAUAAGGCUAGUCCG.

18. The nucleic acid of claim 15, wherein the activator-RNA comprises the 31 nucleotide tracrRNA sequence: UAGCAAGUUAAAAUAAGGCUAGUCCGUUAUC.

19. The nucleic acid of claim 15, wherein the activator-RNA comprises the 64 nucleotide tracrRNA sequence UAGCAAGUUAAAAUAAGGCUAGUCCGUUAUCAACUUGAAAAAGUGGCACCGAGU CGGUGCUUUU.

20. An expression vector comprising a nucleotide sequence encoding a single molecule DNA-targeting RNA, wherein the single molecule DNA-targeting RNA comprises, in 5′ to 3′ order:
(i) a targeter-RNA comprising a first nucleotide sequence that is complementary to a target sequence of a target DNA, and a second nucleotide sequence that hybridizes with an activator-RNA, wherein the first and second nucleotide sequences are heterologous to one another; and
(ii) the activator-RNA, which comprises a duplex-forming segment that comprises a nucleotide sequence that hybridizes with the targeter-RNA to form a double-stranded duplex, wherein (i) and (ii) are covalently linked by intervening nucleotides, and wherein the single molecule DNA-targeting RNA is capable of forming a complex with a Cas9 protein and guiding the complex to the target sequence to cleave the target DNA, wherein the nucleotide sequence encoding the single molecule DNA-targeting RNA is operably linked to a promoter heterologous to the second nucleotide sequence.

21. The expression vector of claim 20, wherein said nucleotide sequence of the duplex-forming segment of the activator-RNA comprises a sequence having 100% identity to the tracrRNA sequence UAGCAAGUUAAAAU.

22. The expression vector of claim 20, wherein the activator-RNA comprises the 26 nucleotide tracrRNA sequence UAGCAAGUUAAAAUAAGGCUAGUCCG.

23. The expression vector of claim 20, wherein the activator-RNA comprises the 31 nucleotide tracrRNA sequence: UAGCAAGUUAAAAUAAGGCUAGUCCGUUAUC.

24. The expression vector of claim 20, wherein the activator-RNA comprises the 64 nucleotide tracrRNA sequence UAGCAAGUUAAAAUAAGGCUAGUCCGUUAUCAACUUGAAAAAGUGGCACCGAGU CGGUGCUUUU.

25. The expression vector of claim 20, wherein the expression vector is a plasmid.

26. A composition comprising
(a) a chimeric Cas9 protein or a nucleic acid encoding the chimeric Cas9 protein, wherein the chimeric Cas9 protein comprises a Cas9 polypeptide fused to a domain for modifying gene expression;
wherein the Cas9 polypeptide is a Streptococcus pyogenes Cas9 with one or more mutations in a RuvC domain and/or an HNH domain; and
(b) a DNA-targeting RNA that comprises:
(i) a targeter-RNA comprising a first nucleotide sequence that is complementary to a target sequence of a target DNA, and a second nucleotide sequence that hybridizes with an activator-RNA; and
(ii) the activator-RNA, which hybridizes with the targeter-RNA to form a double-stranded RNA duplex, wherein (i) and (ii) are covalently linked, wherein the DNA-targeting RNA is capable of forming a complex with the chimeric Cas9 protein and guiding the complex to the target sequence.

27. The composition of claim 26, wherein the Cas9 polypeptide has substantially no nuclease activity.

28. The composition of claim 26, wherein the chimeric Cas9 protein is conjugated, at its carboxyl terminus (C-terminus), to a protein transduction domain (PTD) that is a SV40 nuclear localization signal.

29. The composition of claim 26, comprising the nucleic acid encoding the chimeric Cas9 protein, wherein the nucleic acid encoding the chimeric Cas9 protein is a plasmid.

30. The composition of claim 26, wherein the first nucleotide sequence is 20 or 23 nucleotides long.

31. The composition of claim 26, wherein the activator-RNA comprises the 64 nt tracrRNA sequence UAGCAAGUUAAAAUAAGGCUAGUCCGUUAUCAACUUGAAAAAGUGGCACCGAGU CGGU GCUUUU.

32. The composition of claim 26, wherein the activator-RNA comprises the 26 nucleotide tracrRNA sequence UAGCAAGUUAAAAUAAGGCUAGUCCG.

33. The composition of claim 26, wherein the composition comprises a protein-RNA complex comprising the chimeric Cas9 protein and the DNA-targeting RNA.

34. The composition of claim 26, comprising the DNA-targeting RNA and the nucleic acid encoding the chimeric Cas9 protein.

35. The composition of claim 34, wherein the nucleic acid encoding the chimeric Cas9 protein is a plasmid.

36. The composition of claim 26, further comprising a donor polynucleotide.

37. The composition of claim 26, comprising two or more DNA-targeting RNAs that are capable of hybridizing to different target sequences within the same or different target DNA molecules.

38. A composition comprising
(a) a chimeric Cas9 protein or a nucleic acid encoding the chimeric Cas9 protein, wherein the chimeric Cas9 protein comprises a Cas9 polypeptide fused to a heterologous polypeptide, wherein the Cas9 polypeptide is a Streptococcus pyogenes Cas9 with one or more mutations in a RuvC domain and/or an HNH domain; and
(b) a DNA-targeting RNA comprising:
(i) a targeter-RNA comprising (1) a first nucleotide sequence that is complementary to a target sequence of a target DNA, and (2) a second nucleotide sequence that hybridizes with an activator-RNA; and
(ii) the activator-RNA, which hybridizes with the targeter-RNA to form a double-stranded RNA duplex, wherein (i) and (ii) are covalently linked, wherein the DNA-targeting RNA is capable of forming a complex with the chimeric Cas9 protein and guiding the complex to the target sequence to cleave the target DNA.

39. The composition of claim 38, wherein the Cas9 polypeptide has substantially no nuclease activity.

40. The composition of claim 38, wherein the heterologous polypeptide comprises a transcriptional activator polypeptide or a transcription repressor polypeptide.

41. The composition of claim 40, wherein the Cas9 polypeptide has substantially no nuclease activity.

42. The composition of claim 38, wherein the heterologous polypeptide has histone-modifying activity.

43. The composition of claim 38, wherein the heterologous polypeptide comprises a fluorescent protein.

44. The composition of claim 38, wherein the chimeric Cas9 protein is fused to a nuclear localization domain.

45. The composition of claim 38, wherein the nucleic acid encoding the chimeric Cas9 protein is a plasmid or a vector.

46. The composition of claim 38, wherein the first nucleotide sequence is 20 nucleotides long.

47. The composition of claim 38, wherein the activator-RNA and/or the targeter-RNA comprises a heterologous moiety.

48. The composition of claim 38, wherein the activator-RNA and the targeter-RNA are conjugated to a heterologous moiety.

49. The composition of claim 38, wherein:
(1) the activator-RNA comprises the 64 nt tracrRNA sequence UAGCAAGUUAAAAUAAGGCUAGUCCGUUAUCAACUUGAAAAAGUGGCACCGA GUCGGUGCUUUU;
or (2) the activator RNA comprises the 26 nucleotide tracrRNA sequence UAGCAAGUUAAAAUAAGGCUAGUCCG;
or (3) the targeter-RNA comprises the 12 nucleotide (nt) crRNA sequence GUUUUAGAGCUA and the activator-RNA comprises the 64 nt tracrRNA sequence UAGCAAGUUAAAAUAAGGCUAGUCCGUUAUCAACUUGAAAAAGUGGCACCGA GUCGGUGCUUUU.

50. The composition of claim 38, wherein the composition comprises a protein-RNA complex comprising the chimeric Cas9 protein and the DNA-targeting RNA.

51. The composition of claim 38, comprising the DNA-targeting RNA and the nucleic acid encoding the chimeric Cas9 protein.

52. The composition of claim 51, wherein the nucleic acid encoding the chimeric Cas9 protein is a DNA.

53. The composition of claim 38, further comprising a donor polynucleotide.

54. A composition comprising (a) a chimeric Cas9 protein or a nucleic acid encoding the chimeric Cas9 protein, wherein the chimeric Cas9 protein comprises a Cas9 polypeptide fused to a heterologous polypeptide, wherein the Cas9 polypeptide is a Streptococcus pyogenes Cas9 with one or more mutations in a RuvC domain and/or an HNH domain; and
(b) a DNA-targeting RNA comprising:
(i) a targeter-RNA comprising
(1) a first nucleotide sequence that is complementary to a target sequence of a target DNA, and
(2) a second nucleotide sequence that hybridizes with an activator-RNA; and
(ii) the activator-RNA, which hybridizes with the targeter-RNA to form a double-stranded duplex, wherein (i) and (ii) are covalently linked, wherein the activator-RNA comprises the 64 nt tracrRNA sequence UAGCAAGUUAAAAUAAGGCUAGUCCGUUAUCAACUUGAAAAAGUGGCACCGAGU CGGU GCUUUU, and wherein the DNA-targeting RNA is capable of forming a complex with the chimeric Cas9 protein and guiding the complex to the target sequence to cleave the target DNA.

55. The composition of claim 54, wherein the Cas9 polypeptide has substantially no nuclease activity.

56. The composition of claim 54, wherein said first sequence is 20 nucleotides long and the DNA-targeting RNA comprises the nucleotide sequence GUUUUAGAGCUAGAAAUAGCAAGUUAAAAUAAGGCUAGUCCGUUAUCAACUUGA AAAA GUGGCACCGAGUCGGUGCUUUU.

57. The composition of claim 54, further comprising a donor polynucleotide.

58. A composition comprising
(a) a chimeric Cas9 protein or a nucleic acid encoding the chimeric Cas9 protein,
wherein the chimeric Cas9 protein comprises a Cas9 polypeptide fused to a heterologous polypeptide,
wherein the Cas9 polypeptide is a Streptococcus pyogenes Cas9 with one or more mutations in a RuvC domain and/or an HNH domain;
and (b) a DNA-targeting RNA comprising:
(i) a targeter-RNA comprising
(1) a first nucleotide sequence that is complementary to a target sequence of a target DNA, and
(2) a second nucleotide sequence that hybridizes with an activator-RNA; and
(ii) the activator-RNA, which hybridizes with the targeter-RNA to form a double-stranded RNA duplex, wherein the activator-RNA hybridizes with the targeter-RNA to form a total of 9 to 11 base pairs,
wherein (i) and (ii) are covalently linked,
wherein the DNA-targeting RNA is capable of forming a complex with the chimeric Cas9 protein and guiding the complex to the target sequence to cleave the target DNA.

59. The composition of claim 58, wherein the Cas9 polypeptide has substantially no nuclease activity.

60. The composition of claim 58, wherein the heterologous polypeptide comprises a transcriptional activator polypeptide or a transcription repressor polypeptide.

61. The composition of claim 60, wherein the Cas9 polypeptide has substantially no nuclease activity.

62. The composition of claim 58, wherein the heterologous polypeptide has protein-modifying activity, such as histone modification.

63. The composition of claim 62, wherein the Cas9 polypeptide has substantially no nuclease activity.

64. The composition of claim 58, wherein the heterologous polypeptide has histone-modifying activity.

65. The composition of claim 58, wherein the heterologous polypeptide comprises a fluorescent protein.

66. The composition of claim 58, wherein the chimeric Cas9 protein is fused to a protein transduction domain (PTD), such as nuclear localization signal (NLS).

67. The composition of claim 58, wherein the nucleic acid encoding the chimeric Cas9 protein is a plasmid or a vector.

68. The composition of claim 58, wherein the first nucleotide sequence is 20 or 23 nucleotides long.

69. The composition of claim 58, wherein the activator-RNA and/or the targeter-RNA comprises a heterologous moiety.

70. The composition of claim 58, wherein the activator-RNA and the targeter-RNA are conjugated to a heterologous moiety.

71. The composition of claim 58, wherein the composition comprises a protein-RNA complex comprising the chimeric Cas9 protein and the DNA-targeting RNA.

72. The composition of claim 58, comprising the DNA-targeting RNA and the nucleic acid encoding the chimeric Cas9 protein.

73. The composition of claim 72, wherein the nucleic acid encoding the chimeric Cas9 protein is a DNA.

74. The composition of claim 58, further comprising a donor polynucleotide.

75. A composition comprising
(a) a chimeric Cas9 protein or a nucleic acid encoding the chimeric Cas9 protein,
wherein the chimeric Cas9 protein comprises a Cas9 polypeptide fused to a heterologous polypeptide,
wherein the Cas9 polypeptide is a Streptococcus pyogenes Cas9 with one or more mutations in a RuvC domain and/or an HNH domain; and
(b) a DNA-targeting RNA comprising:
(i) a targeter-RNA comprising
(1) a first nucleotide sequence that is complementary to a target sequence of a target DNA, and
(2) a second nucleotide sequence that hybridizes with an activator-RNA; and
(ii) the activator-RNA, which hybridizes with the targeter-RNA to form a double-stranded RNA duplex, wherein the activator-RNA hybridizes with the targeter-RNA to form a total of 9 to 11 base pairs,
wherein (i) and (ii) are covalently linked,
wherein the DNA-targeting RNA is capable of forming a complex with the chimeric Cas9 protein and guiding the complex to the target sequence to cleave the target DNA.

76. The composition of claim 75, wherein the Cas9 polypeptide has substantially no nuclease activity.

77. The composition of claim 75, wherein the heterologous polypeptide comprises a transcriptional activator polypeptide or a transcription repressor polypeptide.

78. The composition of claim 77, wherein the Cas9 polypeptide has substantially no nuclease activity.

79. The composition of claim 75, wherein the heterologous polypeptide has protein-modifying activity, such as histone modification.

80. The composition of claim 79, wherein the Cas9 polypeptide has substantially no nuclease activity.

81. The composition of claim 75, wherein the heterologous polypeptide has histone-modifying activity.

82. The composition of claim 75, wherein the heterologous polypeptide comprises a fluorescent protein.

83. The composition of claim 75, wherein the chimeric Cas9 protein is fused to a protein transduction domain (PTD), such as a nuclear localization signal (NLS).

84. The composition of claim 75, wherein the nucleic acid encoding the chimeric Cas9 protein is a plasmid or a vector.

85. The composition of claim 75, wherein the first nucleotide sequence is 20 or 23 nucleotides long.

86. The composition of claim 75, wherein the activator-RNA and/or the targeter-RNA comprises a heterologous moiety.

87. The composition of claim 75, wherein the activator-RNA and the targeter-RNA are conjugated to a heterologous moiety.

88. The composition of claim 75, wherein the composition comprises a protein-RNA complex comprising the chimeric Cas9 protein and the DNA-targeting RNA.

89. The composition of claim 75, comprising the DNA-targeting RNA and the nucleic acid encoding the chimeric Cas9 protein.

90. The composition of claim 89, wherein the nucleic acid encoding the chimeric Cas9 protein is a DNA.

91. The composition of claim 75, further comprising a donor polynucleotide.

92. A method of modifying a eukaryotic cell, the method comprising:
providing to a eukaryotic cell
(a) a Cas9 protein; and
(b) a single-molecule DNA-targeting RNA comprising, in 5′ to 3′ order a DNA-targeting segment that comprises a 20 nucleotide long targeting sequence that is complementary to and hybridizes with a target sequence in a chromosomal target DNA of the eukaryotic cell; and a protein-binding segment that is about 80 nucleotides long, interacts with the Cas9 protein, and comprises two complementary stretches of nucleotides that are covalently linked by intervening nucleotides, wherein said complementary stretches of nucleotides hybridize to one another to form a double stranded RNA duplex,
wherein the single-molecule DNA-targeting RNA forms a complex with the Cas9 protein and guides the complex to said target sequence, and the Cas9 protein cleaves the chromosomal target DNA.

93. A method of modifying a eukaryotic cell, the method comprising:
providing to a eukaryotic cell
(a) a Cas9 protein; and
(b) a single-molecule DNA-targeting RNA comprising a 100 nucleotide (nt) sequence that comprises, in 5′ to 3′ order a 20 nucleotide (nt) targeting sequence that is complementary to and hybridizes with a target sequence in a chromosomal target DNA of the eukaryotic cell; a 12 nt crRNA sequence GUUUUAGAGCUA, a 4 nt linker sequence GAAA, and a 64 nt tracrRNA sequence UAGCAAGUUAAAAUAAGGCUAGUCCGUUAUCAACUUGAAAAAGUGGCACCGAGU CGG UGCUUUU,
wherein the single-molecule DNA-targeting RNA forms a complex with the Cas9 protein and guides the complex to said target sequence, and the Cas9 protein cleaves the chromosomal target DNA.

94. The method of claim 92, wherein the eukaryotic cell is provided with the single-molecule DNA-targeting RNA by introducing into the eukaryotic cell a nucleic acid encoding the single-molecule DNA-targeting RNA, and wherein the eukaryotic cell is provided with the Cas9 protein by introducing into the eukaryotic cell a nucleic acid encoding the Cas9 protein.

95. The method of claim 93, wherein the eukaryotic cell is provided with the single-molecule DNA-targeting RNA by introducing into the eukaryotic cell a nucleic acid encoding the single-molecule DNA-targeting RNA, and wherein the eukaryotic cell is provided with the Cas9 protein by introducing into the eukaryotic cell a nucleic acid encoding the Cas9 protein.

96. The method of claim 94, wherein the nucleic acid encoding the single-molecule DNA-targeting RNA or the nucleic acid encoding the Cas9 protein is introduced into the cell using a viral vector.

97. The method of claim 95, wherein the nucleic acid encoding the single-molecule DNA-targeting RNA or the nucleic acid encoding the Cas9 protein is introduced into the cell using a viral vector.

98. The method of claim 94, wherein the nucleic acid encoding the single-molecule DNA-targeting RNA is introduced into the cell using an adeno-associated virus.

99. The method of claim 95, wherein the nucleic acid encoding the single-molecule DNA-targeting RNA is introduced into the cell using an adeno-associated virus.

100. The method of claim 92, wherein the eukaryotic cell is a yeast cell, a plant cell, or a mammalian cell.

101. The method of claim 93, wherein the eukaryotic cell is a yeast cell, a plant cell, or a mammalian cell.

102. The method of claim 92, wherein the eukaryotic cell is a human induced pluripotent stem cell.

103. The method of claim 93, wherein the eukaryotic cell is a human induced pluripotent stem cell.

104. The method of claim 92, comprising providing to the eukaryotic cell two or more of said single-molecule DNA-targeting RNAs, wherein the two or more single-molecule DNA-targeting RNAs each hybridize with different target sequences within said chromosomal target DNA.

105. The method of claim 93, comprising providing to the eukaryotic cell two or more of said single-molecule DNA-targeting RNAs, wherein the two or more single-molecule DNA-targeting RNAs each hybridize with different target sequences within said chromosomal target DNA.

106. The method of claim 104, wherein the eukaryotic cell is provided with the two or more single-molecule DNA-targeting RNAs by introducing into the eukaryotic cell nucleic acids encoding them.

107. The method of claim 105, wherein the eukaryotic cell is provided with the two or more single-molecule DNA-targeting RNAs by introducing into the eukaryotic cell nucleic acids encoding them.

108. The method of claim 92, wherein the single-molecule DNA-targeting RNA is expressed in the eukaryotic cell using a human U6 polymerase III promoter.

109. The method of claim 93, wherein the single-molecule DNA-targeting RNA is expressed in the eukaryotic cell using a human U6 polymerase III promoter.

110. The method of claim 92, wherein said providing results in deletion or insertion of sequence from the chromosomal target DNA.

111. The method of claim 93, wherein said providing results in deletion or insertion of sequence from the chromosomal target DNA.

112. The method of claim 92, wherein the Cas9 protein is fused at its carboxyl terminus (C-terminus) to a protein transduction domain (PTD), wherein said PTD aids in traversal of the Cas9 protein to a nucleus.

113. The method of claim 93, wherein the Cas9 protein is fused at its carboxyl terminus (C-terminus) to a protein transduction domain (PTD), wherein said PTD aids in traversal of the Cas9 protein to a nucleus.

114. The method of claim 92, wherein the Cas9 protein is fused with a C-terminal SV40 nuclear localization sequence.

115. The method of claim 93, wherein the Cas9 protein is fused with a C-terminal SV40 nuclear localization sequence.

116. The method of claim 92, wherein the cleavage of the chromosomal target DNA by the Cas9 protein results in altered expression of the target sequence.

117. The method of claim 93, wherein the cleavage of the chromosomal target DNA by the Cas9 protein results in altered expression of the target sequence.

118. The method of claim 92, wherein the cleavage of the chromosomal target DNA by the Cas9 protein results in nonhomologous end joining.

119. The method of claim 93, wherein the cleavage of the chromosomal target DNA by the Cas9 protein results in nonhomologous end joining.

120. The method of claim 92, further comprising providing a donor polynucleotide to the eukaryotic cell, wherein a sequence of the donor polynucleotide is integrated into the chromosomal target DNA.

121. The method of claim 93, further comprising providing a donor polynucleotide to the eukaryotic cell, wherein a sequence of the donor polynucleotide is integrated into the chromosomal target DNA.

122. A method of modifying a eukaryotic cell, the method comprising:
providing to a eukaryotic cell
(a) a Cas9 protein; and
(b) a first single-molecule DNA-targeting RNA and a second single-molecule DNA-targeting RNA, wherein each single-molecule DNA-targeting RNA comprises, in 5′ to 3′ order a DNA-targeting segment that comprises a 20 nucleotide long targeting sequence; and a protein-binding segment that is about 80 nucleotides long, interacts with the Cas9 protein, and comprises two complementary stretches of nucleotides that are covalently linked by intervening nucleotides, wherein said complementary stretches of nucleotides hybridize to one another to form a double stranded RNA duplex,
wherein the targeting sequence of the first single-molecule DNA-targeting RNA hybridizes with a first target sequence in a chromosomal target DNA of the eukaryotic cell and the targeting sequence of the second single-molecule DNA-targeting RNA hybridizes with a second target sequence in the chromosomal target DNA, and wherein the Cas9 protein cleaves the chromosomal target DNA at the first and second target sequences, resulting in deletion of an intervening fragment of the chromosomal target DNA.

123. A method of modifying a eukaryotic cell, the method comprising:
providing to a eukaryotic cell
(a) a Cas9 protein; and
(b) a first single-molecule DNA-targeting RNA and a second single-molecule DNA-targeting RNA, wherein each single-molecule DNA-targeting RNA comprises a 103 100 nucleotide (nt) sequence that comprises, in 5′ to 3′ order a 20 nucleotide (nt) targeting sequence that is complementary to and hybridizes with a target sequence in a chromosomal target DNA of the eukaryotic cell;
a 12 nt crRNA sequence GUUUUAGAGCUA,
a 4 nt linker sequence GAAA, and
a 64 nt tracrRNA sequence UAGCAAGUUAAAAUAAGGCUAGUCCGUUAUCAACUUGAAAAAGUGGCACCGAGU CGG UGCUUUU,
wherein the targeting sequence of the first single-molecule DNA-targeting RNA hybridizes with a first target sequence in a chromosomal target DNA of the eukaryotic cell and the targeting sequence of the second single-molecule DNA-targeting RNA hybridizes with a second target sequence in the chromosomal target DNA, and wherein the Cas9 protein cleaves the chromosomal target DNA at the first and second target sequences, resulting in deletion of an intervening fragment of the chromosomal target DNA.

124. The method of claim 122, wherein the first and second single-molecule DNA-targeting RNAs are provided by introducing into the eukaryotic cell a nucleic acid encoding the first single-molecule DNA-targeting RNA and a nucleic acid encoding the second single-molecule DNA-targeting RNA, and wherein the Cas9 protein is provided by introducing into the eukaryotic cell a nucleic acid that comprises a nucleotide sequence encoding the Cas9 protein.

125. The method of claim 123, wherein the first and second single-molecule DNA-targeting RNAs are provided by introducing into the eukaryotic cell a nucleic acid encoding the first single-molecule DNA-targeting RNA and a nucleic acid encoding the second single-molecule DNA-targeting RNA, and wherein the Cas9 protein is provided by introducing into the eukaryotic cell a nucleic acid that comprises a nucleotide sequence encoding the Cas9 protein.

126. The method of claim 122, wherein the eukaryotic cell is a yeast cell, a plant cell, a non-human mammalian cell, or a human cell.

127. The method of claim 123, wherein the eukaryotic cell is a yeast cell, a plant cell, a non-human mammalian cell, or a human cell.

128. The method of claim 124, wherein the nucleotide sequence encoding the Cas9 protein is modified to replace one or more codons of a wild-type Cas9 encoding nucleotide sequence with one or more different codons encoding the same amino acid.

129. The method of claim 125, wherein the nucleotide sequence encoding the Cas9 protein is modified to replace one or more codons of a wild-type Cas9 encoding nucleotide sequence with one or more different codons encoding the same amino acid.

130. The method of claim 122, wherein the Cas9 protein is fused to a protein transduction domain (PTD), wherein said PTD aids in traversal of the Cas9 protein from cytosol to within an organelle, wherein the PTD is SV40 nuclear localization signal.

131. The method of claim 123, wherein the Cas9 protein is fused to a protein transduction domain (PTD), wherein said PTD aids in traversal of the Cas9 protein from cytosol to within an organelle, wherein the PTD is SV40 nuclear localization signal.

132. The method of claim 122, wherein the eukaryotic cell is an induced pluripotent stem cell.

133. The method of claim 123, wherein the eukaryotic cell is an induced pluripotent stem cell.

134. A composition, the composition comprising:
(a) a Cas9 protein or a nucleic acid comprising a nucleotide sequence encoding the Cas9 protein; and
(b) a single-molecule DNA-targeting RNA or a nucleic acid comprising a nucleotide sequence encoding the single-molecule DNA-targeting RNA, wherein the single-molecule DNA-targeting RNA comprises, in 5′ to 3′ order
a DNA-targeting segment comprising a 20 nucleotide long targeting sequence that is complementary to a target sequence in a target DNA within a eukaryotic cell; and
a protein-binding segment that is capable of interacting with the Cas9 protein, and
wherein the single-molecule DNA-targeting RNA comprises two complementary stretches of nucleotides that are covalently linked by intervening nucleotides, wherein said complementary stretches of nucleotides hybridize to one another to form a stem-loop structure,
wherein the single-molecule DNA-targeting RNA is capable of forming a complex with the Cas9 protein and guiding the complex to said target sequence.

135. A composition comprising:
(a) a Cas9 protein or a nucleic acid encoding the Cas9 protein; and
(b) a single-molecule DNA-targeting RNA or a nucleic acid encoding the single-molecule DNA-targeting RNA, wherein the single-molecule DNA-targeting RNA comprises a nucleotide (nt) sequence that comprises, in 5′ to 3′ order
a 20 nucleotide (nt) targeting sequence that is complementary to a target sequence in a target DNA within a eukaryotic cell;
a 12 nt crRNA sequence GUUUUAGAGCUA, a 4 nt linker sequence GAAA, and a 64 nt tracrRNA sequence UAGCAAGUUAAAAUAAGGCUAGUCCGUUAUCAACUUGAAAAAGUGGCACCGAGU CG GUGCUUUU, wherein the single-molecule DNA-targeting RNA is capable of forming a complex with the Cas9 protein and guiding the complex to said target sequence.

136. The composition of claim 134, wherein the single-molecule DNA-targeting RNA comprises the 26 nucleotide tracrRNA sequence UAGCAAGUUAAAAUAAGGCUAGUCCG.

137. An ex vivo eukaryotic cell containing the composition of claim 134.

138. An ex vivo eukaryotic cell containing the composition of claim 135.

139. The eukaryotic cell of claim 137, wherein the eukaryotic cell is a yeast cell, a plant cell, a mammalian cell, or a human cell.

140. The eukaryotic cell of claim 138, wherein the eukaryotic cell is a yeast cell, a plant cell, a mammalian cell, or a human cell.

141. The eukaryotic cell of claim 137, wherein the eukaryotic cell is a human induced pluripotent stem cell.

142. The eukaryotic cell of claim 138, wherein the eukaryotic cell is a human induced pluripotent stem cell.

143. The composition of claim 134, wherein the nucleotide sequence encoding the single-molecule DNA-targeting RNA is operably linked to a regulatory element that is operable in the eukaryotic cell.

144. The composition of claim 135, wherein the nucleotide sequence encoding the single-molecule DNA-targeting RNA is operably linked to a regulatory element that is operable in the eukaryotic cell.

145. The composition of claim 134, wherein the nucleotide sequence encoding the single-molecule DNA-targeting RNA is operably linked to a human U6 polymerase III promoter.

146. The composition of claim 135, wherein the nucleotide sequence encoding the single-molecule DNA-targeting RNA is operably linked to a human U6 polymerase III promoter.

147. The composition of claim 134, wherein the nucleotide sequence encoding the Cas9 protein is modified to replace one or more codons of a wild-type Cas9 encoding nucleotide sequence with one or more different codons encoding the same amino acid.

148. The composition of claim 135, wherein the nucleotide sequence encoding the Cas9 protein is modified to replace one or more codons of a wild-type Cas9 encoding nucleotide sequence with one or more different codons encoding the same amino acid.

149. The composition of claim 147, wherein the Cas9 protein is fused at its carboxyl terminus (C-terminus) to a protein transduction domain (PTD), wherein said PTD aids in traversal of the Cas9 protein from cytosol to within an organelle, wherein the PTD is SV40 nuclear localization signal.

150. The composition of claim 148, wherein the Cas9 protein is fused at its carboxyl terminus (C-terminus) to a protein transduction domain (PTD), wherein said PTD aids in traversal of the Cas9 protein from cytosol to within an organelle, wherein the PTD is SV40 nuclear localization signal.

151. The composition of claim 147, wherein the nucleotide sequence encoding the Cas9 protein is operably linked to a regulatory element that is operable in the eukaryotic cell.

152. The composition of claim 148, wherein the nucleotide sequence encoding the Cas9 protein is operably linked to a regulatory element that is operable in the eukaryotic cell.

153. The composition of claim 134, wherein the eukaryotic cell is a yeast cell, a plant cell, a mammalian cell, or a human cell.

154. The composition of claim 135, wherein the eukaryotic cell is a yeast cell, a plant cell, a mammalian cell, or a human cell.

155. The composition of claim 134, wherein the eukaryotic cell is a human induced pluripotent stem cell.

156. The composition of claim 135, wherein the eukaryotic cell is a human induced pluripotent stem cell.

157. The composition of claim 134, wherein the eukaryotic cell is a yeast cell, a plant cell, a mammalian cell, or a human cell.

158. The composition of claim 135, wherein the eukaryotic cell is a yeast cell, a plant cell, a mammalian cell, or a human cell.

159. The composition of claim 134, wherein the eukaryotic cell is a human induced pluripotent stem cell.

160. The composition of claim 135, wherein the eukaryotic cell is a human induced pluripotent stem cell.

161. An ex vivo eukaryotic cell comprising:
(a) a Cas9 protein or a nucleic acid comprising a nucleotide sequence encoding the Cas9 protein; and
(b) a single-molecule DNA-targeting RNA or a nucleic acid comprising a nucleotide sequence encoding the single-molecule DNA-targeting RNA, wherein the single-molecule DNA-targeting RNA comprises, in 5′ to 3′ order
a DNA-targeting segment comprising a 20 nucleotide long targeting sequence that is complementary to and capable of hybridizing with a target sequence in a target DNA within the eukaryotic cell; and
a protein-binding segment that is capable of interacting with the Cas9 protein, and comprises two complementary stretches of nucleotides that are covalently linked by intervening nucleotides, wherein said complementary stretches of nucleotides hybridize to one another to form a stem-loop structure, wherein the single-molecule DNA-targeting RNA is capable of forming a complex with the Cas9 protein and guiding the complex to said target sequence,
and wherein the eukaryotic cell is a stem cell.

162. An ex vivo eukaryotic cell comprising:
(a) a Cas9 protein or a nucleic acid comprising a nucleotide sequence encoding the Cas9 protein; and
(b) a single-molecule DNA-targeting RNA or a nucleic acid comprising a nucleotide sequence encoding the single-molecule DNA-targeting RNA,
wherein the single-molecule DNA-targeting RNA comprises a nucleotide (nt) sequence that comprises, in 5′ to 3′ order a 20 nucleotide (nt) targeting sequence that is complementary to and capable of hybridizing with a target sequence in a target DNA within the eukaryotic cell;
a 12 nt crRNA sequence GUUUUAGAGCUA, a 4 nt linker sequence GAAA, and a 64 nt tracrRNA sequence UAGCAAGUUAAAAUAAGGCUAGUCCGUUAUCAACUUGAAAAAGUGGCACC GAGUCGGUGCUUUU, wherein the single-molecule DNA-targeting RNA is capable of forming a complex with the Cas9 protein and guiding the complex to said target sequence, and wherein the eukaryotic cell is a stem cell.

163. The ex vivo eukaryotic cell of claim 161, wherein the stem cell is a human induced pluripotent stem cell.

164. The ex vivo eukaryotic cell of claim 162, wherein the stem cell is a human induced pluripotent stem cell.

165. A single-molecule DNA-targeting RNA comprising, in 5′ to 3′ order:
a targeting sequence that is complementary to a target DNA sequence within a eukaryotic cell;
GUUUUAGAGCUA, which is a crRNA sequence;
GAAA, which is a linker sequence; and
UAGCAAGUUAAAAUAAGGCUAGUCCGUUAUCAACUUGAAAAAGUGGCACC GAGUC GGUGCUUUU, which is a 64 nt tracrRNA sequence.
